(19) United States
(12) Patent Application Publication
(10) Pub. No.: US 2023/0267369 A1
(43) Pub. Date: Aug. 24, 2023

(54) GENERALIZED LOCAL ADAPTIVE FUSION REGRESSION PROCESS BASED ON PHYSICOCHEMICAL AND PHYSIOCHEMICAL UNDERLYING HIDDEN PROPERTIES FOR QUANTITATIVE ANALYSIS OF MOLECULAR BASED SPECTROSCOPIC DATA

(71) Applicant: IDAHO STATE UNIVERSITY, Pocatello, ID (US)

(72) Inventors: JOHN KALIVAS, POCATELLO, ID (US); ROBERT SPIERS, BOISE, ID (US)

(21) Appl. No.: 18/072,090

(22) Filed: Nov. 30, 2022

Related U.S. Application Data
(60) Provisional application No. 63/304,856, filed on Jan. 31, 2022.

(51) Int. Cl. G06N 20/00; G16B 40/20

(52) U.S. Cl. CPC G06N 20/00; G16B 40/20

(57) ABSTRACT

Methodologies and corresponding systems for a Local adaptive fusion regression (LAFR) process are able to search a large library of spectral measurement for a linear calibration (training) set, which is spectrally matrix matched to a target sample spectrum, and also tightly bracketed about an “unknown” prediction property (analyte) for the target sample. Using a matched calibration set, the likelihood of an accurate prediction by the selected calibration set is greatly enhanced. The LAFR process integrates multiple spectral similarity information with contextual considerations between source analyte contents, model, and analyte predictions. LAFR facilitates onsite chemical analysis such as with a handheld spectrometer, dedicated in-line process analyzers and benchtop instruments. LAFR is based on a Beer’s law like linear relationship where a calibration model (mathematical relationship) is made that linearly relates the analyte amount, e.g., concentration, to the measured spectral responses. The calibration model is then used to predict (quantitate) the analyte amounts present in new samples.

### PRIORITY CLAIM

The present application claims the benefit of priority of U.S. Provisional Pat. Application No. 63/304,856, titled “Generalized Local Adaptive Fusion Regression Process Based on Physicochemical and Physiochemical Underlying Hidden Properties for Quantitative Analysis of Molecular Based Spectroscopic Data,” filed Jan. 31, 2022, which is fully incorporated herein by reference for all purposes.

### STATEMENT REGARDING SPONSORED RESEARCH OR DEVELOPMENT

The presently disclosed subject matter was made with Government support under Grant Nos. CHE-1506417 and CHE-1904166, awarded by the National Science Foundation. The Government has certain rights in the invention.

### BACKGROUND OF THE PRESENTLY DISCLOSED SUBJECT MATTER

The local adaptive fusion regression (LAFR) algorithm is designed to facilitate onsite chemical analysis with a handheld spectrometer and by default, dedicated in-line process analyzers and benchtop instruments. It is an understandable process based on a Beer’s law-like linear relationship where a calibration model (mathematical relationship) is made that linearly relates the analyte amount, e.g., concentration, to the measured spectral responses. The calibration model is then used to predict (quantitate) the analyte amounts present in new samples. Unlike other calibration methods, LAFR takes advantage of unique features present in chemical spectral data where each measured sample spectrum stems from not only the analyte property amount but from all other spectrally responding factors responsible for the sample measured values. These factors include the sample-wise unique molecular relationships relative to the respective physicochemical, and if applicable, physiochemical conditions, measurement conditions, and instrumentation effects. The composite of these measurement influences goes by many names, but in analytical chemistry, it is termed the sample matrix effects. The usual chemical analysis approach by calibration is to always correct for the matrix effects, thereby causing current calibration methods to fail in new sample situations. The LAFR thesis is to instead use the inherent matrix effects as information for training sample sets (calibration samples) to successfully predict new samples. Matrix effects can be considered hidden variables, and thus, are impossible to explicitly detail. The LAFR algorithm searches (mines) through a library of spectral samples with corresponding analyte values and identifies subsets of samples with similar matrix effects. No other algorithm is currently able to do this with chemical data. The objective of LAFR is for the final local training set to be composed of samples with the analyte amount highly similar to the unknown analyte amount in the target sample needing prediction, i.e., calibration sample analyte amounts closely bracket the unknown target sample analyte amount. Because the target sample analyte amount is not known, the inability of other methods to identify calibration samples accomplishing the LAFR objective is why they fail.

For LAFR to successfully identify matrix-matched samples, a novel computational tool termed indicator of system uniqueness (ISU) was developed to assess the degree of matrix matching between reference samples and the target sample. It includes a novel sample-wise difference approach to matrix-match spectra (ISU_{X}) and matrix-match actual and predicted analyte reference amounts (ISU_{y}). It is the ISUy that allows matching the unlabeled (unknown) target sample analyte amount to the known analyte reference samples. Without this measure, other local modeling algorithms fail. The ISU (1) holistically characterizes similarity by fusion of multiple similarity merits; (2) does not require advanced optimization processes; and (3) is used throughout the many LAFR steps. Note, the concept of matching by fusion of similarity measures was used in the first 2015 LAFR disclosure, but the current process is much different now and many similarity measures in the first fusion version have been removed because they were found to be detrimental to LAFR. Additionally, some new similarity measures, also referred to as reliability measures, are now included without which, LAFR could also not succeed.

### SUMMARY OF THE PRESENTLY DISCLOSED SUBJECT MATTER

Aspects and advantages of the presently disclosed subject matter will be set forth in part in the following description, or may be apparent from the description, or may be learned through practice of the presently disclosed subject matter.

Broadly speaking, the presently disclosed subject matter relates in some respects to the LAFR algorithm using the ISU_{X} and ISU_{y} sample-wise similarities to mine a library of field sample spectra with reference amounts for a local training set explicitly matrix-matched to the target prediction sample. A model is formed with this training set and is used to predict the target sample. While it has many adjustable parameters, all parameters are self-optimized.

Since LAFR includes an algorithm, the steps leading to a final local calibration set to form a prediction model for a particular target sample are now outlined:
1. Initiate the algorithm with a sample spectral library spanning a diverse set of physicochemical and/or physiochemical conditions. Each sample has reference values (labels) for the amounts of the desired prediction proprieties (analytes).
2. Measure the spectrum of the target sample to be predicted.
3. Define the LAFR parameter set (a collection of LAFR parameter values).**
4. Select a combination of parameter values from the parameter set suite.
Steps 5 - 10 are relative to the parameter values selected.
5. Reduce the library to a smaller subset spectrally similar to the target based on a fusion of multiple measures.
6. Spectrally outlier checks the reduced sample set and remove samples deemed outliers. Spectrally outlier checks the target sample to the final smaller sample set. If the target sample is an outlier, it is deemed non-predictable relative to the smaller subset. Using a new combination of parameter values from Step 4, form a new smaller subset, and go to Step 5. All outlier checks are performed based on a fusion of multiple outlier measures. Those measures requiring tuning parameter optimization are not optimized, but instead, tuning parameter windows are used.
7. Break the sample set into smaller linear training sets using ISUₓ and ISU_{y}. Note: This critical step was not part of the original LAFR disclosed in 2015. Without this change, the original LAFR was not generalizable across the multitude of matrix effect variants present in spectral data sets and it only worked for one data set with limited matrix effects and few library samples.
8. Outlier checks each training set sample spectrally and by predicted analyte errors and remove outliers. A collection of quality control (QC) measures is now used post outlier cleaning to ensure the training set can make reasonable predictions. If the training set does not pass the QC check, it is removed from further consideration. Without the new QC step, LAFR would fail, and no other local algorithm uses this QC step.
9. Outlier checks the target sample to each training set (1) spectrally and (2) using prediction similarities (unlabeled outlier checking). If the target sample is an outlier, remove that training set from further consideration. Being able to outlier check the target sample based on predicted values is another novel component of LAFR not present in other local modeling algorithms.
10. Using the novel sample-wise difference approach, calculate respective target sample ISUₓ and ISU_{y} values for each linear training set using the novel cross- and self-model approach. Rank and select the best training set by the smallest composite ISU value and store the selected calibration set.
11. If there are other combinations of parameter values, go to Step 4; otherwise continue.
12. Calculate respective target sample ISUₓ and ISU_{y} self-model difference values for each saved linear training set in Step 10. Rank and select best training sets by the smallest composite ISU value.
13. From the final best training sets, use the sample-wise ISUₓ and ISU_{y} values, rank, and select the best matched training samples to form the final training set.
14. Build a model with the final training set and predict the target sample.
15. Based on the final training set, determine the target sample membership value to assess the reliability of the predicted analyte amount in the target sample. This last step is also novel to LAFR.
**NOTE: Steps 11 and 12 can be part of the defined parameter set of values in step 3 for another layer of training set formation and selection.

### Fundamental Parameter Set Variables

Overall
   Number of principal components (PCs)
   Partial Least Squares (PLS) model selection
Library Decimation
   Spectral similarity measures
   Number of samples post-decimation
Outlier Cleaning
   Outlier detection measures
   Number of outliers to remove each iteration
   Whether to remove when checking unlabeled x_{target}
   Self-prediction thresholds
Training Set formation
   Number of calibration sets
   First cluster sample and initial distance measure
   ISUₓ / ISU_{y} / Error weighting on set formation
ISU Matrix Matching
   Similarity measures
   Fusion rule
Set and sample selection
   N training (CSWiM) sets
   K training (SWiM) samples

Handheld spectral devices expand chemical analysis to now be possible onsite, i.e, bringing the laboratory to the sample. However, being able to make spectral measurands in the field is not useful unless a real-time accurate local calibration/prediction method exists. In order to obtain a quantitative prediction of a target sample analyte, the calibration set must properly span the target sample in terms of all hidden matrix effects, e.g., physicochemical properties, relative to both spectral measurements (X) and amounts of all spectral responding factors (Y). This matching constraint confounds automatic calibration and prediction limiting chemical analysis by handheld devices. Local modeling is a framework that produces calibration sets matched to target samples. Local modeling requires mining a library composed of thousands of analyte reference spectra with a vast array of implicit matrix effects with the intent of identifying a unique calibration set specifically matrix matched to the particular target sample being predicted. Rather than forming one local model, the presented approach, termed local adaptive fusion regression (LAFR), forms hundreds of linear local models from a library with each model representing a distinct combination of hidden matrix effects. The LAFR approach considers local modeling as a classification situation where a target sample is classified into the local model with the most similar matrix effects. Unique to LAFR are many new concepts not used in any other local modeling method currently available. Key innovations are the indicator of system uniqueness (ISU), a hybrid fusion algorithm to characterize X and Y similarities including sample-wise differences, and each sample prediction amount receives a membership value relative to the predicting calibration set.

The computer algorithm and device allows any person, including the typical consumer, with an appropriate handheld measuring device to perform chemical analysis for the amount of substance present in a sample. Such a capability allows the person to perform the chemical analysis in the field. The algorithm will also perform equally well with a laboratory-based device.

There are plenty of computer algorithms that perform quantitative chemical analysis with handheld and laboratory based instruments. Typical current jargon is “machine learning” and “artificial intelligence.” These algorithms require big data type training data sets to build prediction models. Where all local modeling algorithms fail is when the chemical analysis is performed on new samples that are slightly or greatly different from the training set. Unfortunately, this predicament is the usual situation. The LAFR algorithm solves the problem stifling local modeling from going forward to real world applications. Unique to the algorithm is the ability to identify from a big data library base, those reference samples best mimicking the new sample requiring quantitative analysis. No other algorithm can accomplish this. With this key sample set identified, it becomes the training set to form the model to predict the particular sample.

As with all local modeling algorithms, the process is repeated for each new sample. With LAFR in hand, it now becomes possible for immediate onsite analysis with a handheld device. For example, forest personal could quickly characterize the health or net worth of trees by for example the pulp content. Another example is a farmer could immediately assess the nutrient amounts in their soil or a rancher could instantly assess the health of their livestock from their feces. Lastly, it may be possible to now finally solve the multi-decades old search for an algorithm that would allow non-invasive glucose monitoring, i.e., without any requirement of blood samples or implanted electrodes. The potential uses of LAFR are abundant.

A large market for LAFR is the everyday consumer where LAFR would make it possible for the consumer to perform immediate chemical analysis on the spot. The algorithm may also be applicable to non-invasive glucose monitoring, thereby expanding the market. Industries requiring onsite chemical analysis with handheld devices or using dedicated inline sensors could use LAFR such as agriculture and pharmaceutical applications.

With a large enough data base and a purchased spectrometer, access to the LAFR algorithm would be regulated by a smartphone app with a charge for each analysis depending on the product design. With a large library, the costly expense of sending samples to a laboratory for analysis would no longer be needed and results would essentially be instantaneous. With LAFR, the time expense for a wet chemical laboratory-based analysis would be eliminated. Currently, there is a large enough NIR soil library that agriculture would be the first target area.

A fast and efficient strategy is proposed – the representative approach – for big data analysis with generalized linear models, especially for distributed data with localization requirements or limited network bandwidth. With a given partition of massive dataset, this approach constructs a representative data point for each data block and fits the target model using the representative dataset. In terms of time complexity, it is as fast as the subsampling approaches in the literature. As for efficiency, its accuracy in estimating parameters given a homogeneous partition is comparable with the divide-and-conquer method. Supported by comprehensive simulation studies and theoretical justifications, we conclude that mean representatives (MR) work fine for linear models or generalized linear models with a flat inverse link function and moderate coefficients of continuous predictors. For general cases, we recommend the proposed score-matching representatives (SMR), which may improve the accuracy of estimators significantly by matching the score function values.

Considered another way, presently disclosed methodologies and corresponding systems for a local adaptive fusion regression (LAFR) process are able to search (data mine) a large library of spectral measurement (such as near infrared (NIR), Raman, nuclear magnetic resonance (NMR), or another form sample of collected measurements) for a linear calibration (training) set. The training set is not only spectrally matrix matched to a target sample spectrum, but also tightly brackets the “unknown” prediction property (analyte) for the target sample. Using a matched calibration set, the likelihood of an accurate prediction by the selected calibration set is greatly enhanced. The LAFR process integrates multiple spectral similarity information with contextual considerations between source analyte contents, model, and analyte predictions. LAFR facilitates onsite chemical analysis such as with a handheld spectrometer, dedicated in-line process analyzers and benchtop instruments. LAFR is based on a Beer’s law-like linear relationship where a calibration model (mathematical relationship) is made that linearly relates the analyte amount, e.g., concentration, to the measured spectral responses. The calibration model is then used to predict (quantitate) the analyte amounts present in new samples.

In one exemplary embodiment disclosed herewith, a methodology for searching a large library of field sample spectra (Library X, y) uses a generalized local adaptive fusion regression (LAFR) process for quantitative analysis of molecular-based spectroscopic data (x_{new}) from a target sample of analytes. Such methodology preferably comprises defining LAFR process parameters, including the number of library samples to retain in a decimation step, the number of calibration clusters to form, and the number of fundamental parameters to use; applying a decimation step to the library to reduce the library to most N spectrally similar to target sample, and to perform an outlier check to remove reduced library components for which the target sample is an outlier; forming linear calibration sets defined by the LAFR process parameters; performing an outlier check to remove linear calibration sets for which the target sample is an outlier; using ISUₓ and ISU_{y} sample-wise similarities to mine the library of field sample spectra with reference amounts for a local training set explicitly matrix-matched to the target prediction sample; forming a prediction model formed with the local training set; and using the prediction model to predict the quantitative analysis of a target sample. Preferably per such methodology, ISUₓ and ISU_{y} sample-wise similarities comprise indicators of system uniqueness (ISU) to assess the degree of matrix matching between reference samples and the target sample.

Another exemplary embodiment disclosed herewith relates to methodology for searching a large library of spectral measurement (Library X, y) using a generalized local adaptive fusion regression (LAFR) process for quantitative analysis of molecular based spectroscopic data (x_{new}) from a target sample of analytes. Such methodology preferably comprises defining process parameters and obtaining all possible hyperparameter combinations (HPPC’s) thereof; for each HPPC: reduce Library to N most spectrally similar to the target sample; form calibration sets (CalSets) by clustering analyte ranged windows; remove all CalSets for which the target is an outlier to produce approved CalSets; use matrix matching to identify a selected CalSet from the approved CalSets which best matches to the target sample; store the selected CalSet for each HPPC; use matrix matching to select best N sets from the stored Selected CalSets; use matrix matching to select best K samples from the N selected CalSets; form a calibration model with the K samples; and apply the LAFR process to a new target sample to predict the analysis thereof.

Yet another exemplary embodiment disclosed herewith relates to methodology for predicting the quantitative analysis of a target sample, preferably comprising searching through a library of spectral samples with corresponding analyte values (Library X, y) using a generalized local adaptive fusion regression (LAFR) process for identifying subsets of samples with similar matrix effects; forming linear training sets defined by the LAFR process identified subsets of samples; forming a final local training set from the linear training sets; forming a prediction model with the final local training set; and using the prediction model to predict the quantitative analysis of a target sample, where the final local training set is composed of samples with the analyte amount highly similar to the unknown analyte amount in the target sample to be predicted.

It is to be understood that the presently disclosed subject matter equally relates to associated and/or corresponding apparatuses

Other example aspects of the present disclosure are directed to systems, apparatus, tangible, non-transitory computer-readable media, user interfaces, memory devices, and electronic devices for analysis of molecular based spectroscopic data. To implement methodology and technology herewith, one or more processors may be provided, programmed to perform the steps and functions as called for by the presently disclosed subject matter, as will be understood by those of ordinary skill in the art.

One presently disclosed exemplary embodiment may relate to a handheld spectral device operating according to any of the methodologies disclosed herewith, for making spectral measurements of a target sample in the field and predicting the quantitative analysis thereof. Per some embodiments there, such handheld device may comprise a smartphone remotely accessing an app for operating according to any of the methodologies disclosed herewith.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed; and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying figures, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates an example of matrix matching from Global Calibration sets with local calibration sets;

FIG. 2 illustrates an example of a known data handling method, such as disclosed by Kalivas, J., Emerson, R.; Taking a big data approach to local spectral calibration;

FIG. 3 illustrates an explained flowchart for the presently disclosed Local Adaptive Fusion Regression (LAFR) process for local modeling and calibration set selection;

FIG. 4(a) illustrates spectra of library and target samples for a present meat-based example;

FIGS. 4(b) and 4(c) illustrate histograms of Fat and Protein concentrations, respectively, for both the library and target samples of FIG. 4(a);

FIG. 5(a) graphically illustrates Global prediction and LAFR prediction of fat content for each sample, with the CalSet analyte distribution boxplots (upper), relating to the dataset associated with FIG. 4(a);

FIG. 5(b) graphically illustrates actual and predicted fat content by the LAFR model and the global model for correlation analysis relative to FIG. 5(a) dataset;

FIG. 6(a) graphically illustrates Global prediction and LAFR prediction of meat protein content for each sample, with the CalSet analyte distribution boxplots (upper), relating to the dataset associated with FIG. 4(a);

FIG. 6(b) graphically illustrates actual and predicted protein content by the LAFR model and the global model for correlation analysis relative to FIG. 6(a) dataset;

FIG. 7 diagrammatically illustrates interplay of matrix effects;

FIG. 8 graphically illustrates mean spectra of the corn samples from the three indicated instruments;

FIG. 9 graphically illustrates mean spectra of temperature samples from the five indicated temperatures, as follows: Temperature 16 ethanol/water/isopropanol mixtures measured at five temperatures (30, 40, 50, 60, 70° C.);

FIG. 10 graphically illustrates prediction error of corn samples against their corresponding z-score ISUₓ and ISU_{y} results;

FIG. 11 graphically illustrates average prediction error of temperature datasets against average z-score ISUₓ and ISU_{y} results;

FIG. 12 graphically illustrates average prediction error of temperature datasets against combined ISU_{Xy} Membership Factor achieved by converting the ISUₓ and ISU_{y} to z-table probabilities, then multiplying the probabilities together;

FIG. 13 relates to outlier detection, and graphically illustrates (on the left side thereof) the number of outliers against z-threshold for the three corn datasets as source, individually, and on the right-side thereof, illustrates RMSE (Root Mean Squared Error) of the samples identified as outliers or non-outliers from a particular source calibration set;

FIG. 14 graphically illustrates 240 target samples from corn m5, mp5, and mp6, respectively, compared using the ISU to the three-source calibration sets;

FIG. 15 graphically illustrates a confusion matrix for matrix matching target samples into their actual calibration set of origin;

FIG. 16 graphically illustrates RMSEV (root mean squared error of validation samples) of model updating mechanisms for the source calibration set updating to target conditions, regarding results in the context of calibration transfer;

FIG. 17 represents possible use of just in time learning or so-called lazy learning to address going from a global library to a local condition;

FIG. 18 graphically represents new target samples among a library of historical data;

FIG. 19 graphically represents that the calibration set needs to matrix match each target sample by X, hidden spectral effects;

FIG. 20 graphically represents that the calibration set needs to matrix match each target sample by Y, analyte and unknown interferent amounts.

FIG. 21 graphically represents that models may be from calibration sets with similar implicit matrix effects;

FIG. 22 represents pertinent Local Mean Centering (LMC) and Null Augmented Regression (NAR) approaches;

FIG. 23 graphically illustrates an example of a new sample (diamond);

FIG. 24 graphically illustrates there is a matrix match calibration set X and y to the new target sample, referenced with FIG. 23;

FIGS. 25(a), 25(b), and 25(c) graphically illustrate, respectively, examples of the initial library, then the determined linear clusters within the library, and the interplay with a target sample;

FIG. 26 illustrates mathematical representations of a calibration set (henceforth ignoring e, i.e., random noise), where each calibration sample has unique matrix effects;

FIG. 27 graphically represents treatment of a new target sample, where each new target prediction sample has unique matrix effects;

FIG. 28 is a graphical illustration of outlier detection without tuning parameter selection;

FIG. 29 graphically represents consensus classification using non-optimized classifiers;

FIG. 30 graphically represents self-optimized one-class classification;

FIG. 31 graphically represents leveraging model regression vectors for matrix matching;

FIG. 32 graphically represents the mean of ISU differences across 6 calibration sets;

FIG. 33 graphically represents diagonal blocks which are self-model ISU differences, where x_{new} is matrix matched to primary source calibration set 5;

FIG. 34 graphically represents off-diagonal blocks which are cross-model ISU differences;

FIG. 35 illustrates in block diagram format a flowchart for an overview of the presently disclosed Local Adaptive Fusion Regression (LAFR) process subject matter;

FIG. 36 graphically represents tracking presently disclosed matrix matching functionality going from Global CalSet to Local CalSets, in a “fat content” example;

FIG. 37 is a block diagram illustration of using model selection for multiple tuning parameters;

FIG. 38 graphically and diagrammatically represents selecting models biased to unlabeled target samples;

FIG. 39 graphically illustrates R1 to R2 results, of moisture re soy seed samples, and uses as background resource;

FIG. 40 graphically illustrates target calibration predicting unlabeled target samples, in conjunction with SPS (secondary predicting secondary);

FIG. 41 graphically represents that all null augmented regression (NAR) approaches work equivalently to local mean centering (LMC) at minimum and 1st quartile root mean squared error of validation (RMSEV) (R²);

FIG. 42 graphically illustrates that one may include a few target references to improve NAR at 1^{st} quartile and median;

FIG. 43 graphically represents that MDPS selects models at or below 1st quartile, with target samples used: (1) in MDPS to select NAR and LMC models, and (2) in R to form NAR models;

FIG. 44 represents a summary of Components of NAR with MDPS

FIG. 45 represents the two-step process associated with the subject matter of FIG. 44;

FIGS. 46(a)-46(e) represents automatic field analysis with presently disclosed LAFR and NAR, in a number of areas, representing operation with food, plastics, pharmaceutical, forestry, and wine products, respectively;

FIG. 47 graphically represents the cumulative impacts and/or respective contributions of calibration and prediction on resulting validation;

FIG. 48 graphically illustrates relationship between fit regression and expanded library approaches;

FIG. 49 graphically illustrates identifying a local subset in relation to prediction via local model;

FIG. 50 graphically illustrates identifying a local subset in relation to prediction via local model resulting in different matrix effects;

FIG. 51 diagrammatically illustrates an aspect of the presently disclosed Local Adaptive Fusion Regression (LAFR) process;

FIG. 52 graphically illustrates presently disclosed relation between clustering, regression, and prediction process facets;

FIG. 53 graphically represents, in the context of spectral similarity measures, that outlier detection may be achieved without tuning parameter selection;

FIG. 54 graphically represents that further consensus classification may be achieved using non-optimized classifiers;

FIG. 55 graphically represents that self-optimized one-class classification (class modeling) may be achieved;

FIG. 56 graphically represents, in the context of “analyte” similarity measures, that model regression vectors may be leveraged for matrix matching;

FIGS. 57(a) - 57(d) graphically illustrate data regarding a meat data example, in particular, FIG. 57(a) illustrates spectra from 850 - 1050 nm (100 wavelengths), while FIGS. 57(b) through 57(d), respectively illustrate moisture, fat, and protein;

FIGS. 58(a) and 58(b) graphically represent the global calibration set and the LAFR calibration sets as to fat values;

FIGS. 59(a) and 59(b) graphically represent the LAFR calibration sets locally analyte matched to target, and graphically illustrate that the results of the LAFR predictions are more accurate than global predictions, all in fat content context;

FIGS. 60(a) and 60(b) graphically represent, respectively, the global calibration set and the LAFR calibration sets as to protein values;

FIGS. 61(a) and 61(b) graphically represent the LAFR calibration sets locally analyte matched to target, all in protein content context;

FIG. 62 graphically illustrates comparisons of library data and tests (targets) concerning a soil data-based example;

FIGS. 63(a) and 63(b) graphically represent that calibration sets are highly localized to target analyte, and LAFR predictions are better than global (LAFR predictions strong and much better than global);

FIGS. 64(a) - 64(c) graphically and diagrammatically illustrate US climate regions which tend to comprise US calibration samples, illustrating respectively US regions (FIG. 64(a)), frequency of calibration samples by region (FIG. 64(b)), and fraction of calibration samples relative to region totals (FIG. 64(c)).

FIGS. 65(a) - 65(c) respectively graphically illustrate moisture concentration, wavelength, and principal component (PC) comparisons for such corn-related data.

FIGS. 66(a) - 66(d) graphically illustrate various analytic results, using 30 minutes per sample, with 6 parameter sets, variously comparing as indicated global model data, actual data, and LAFR data;

FIGS. 67(a) - 67(c) graphically illustrate associated wavelength, principal component (PC) comparisons, and protein concentrations data, all for another set of corn data which was considered, with all instruments in Library.

FIGS. 68, 69(a), 69(b), and 70 variously graphically illustrate data respectively associated with moisture values for global calibration set (FIG. 68), protein content for data re Global predicted, actual, and LAFR predicted (FIG. 69(a)), actual versus predicted protein content data (FIG. 69(b)), and m5 versus mp5 versus mp6 comparison data per the target sample instrument (FIG. 70);

FIG. 71(a) graphically illustrates crude protein concentration in library versus target compared frequencies for a present example;

FIG. 71(b) graphically illustrates library versus test data for principal component (PC) comparisons for the example associated with FIG. 71(a);

FIG. 71(c) graphically illustrates the response and wavelength comparisons for library versus test data for the example associated with FIG. 71(a);

FIG. 72(a) graphically illustrates crude protein content for data re Global predicted, actual, and local predicted for a present example;

FIG. 72(b) graphically illustrates actual versus predicted crude protein content data for a present example;

FIG. 73 illustrates a diagrammatical flowchart of the presently-disclosed Local Adaptive Fusion Regression (LAFR) Process, and involving steps of Decimate, Cluster, Classify, and Predict;

FIGS. 74(a) through 74(c) graphically represent the effects of low linearity versus medium linearity versus high linearity, respectively;

FIG. 75 illustrates a diagrammatical flowchart representing how the Local Adaptive Fusion Regression (LAFR) Process steps Decimate/Cluster/Classify/Predict can be considered over n different possibilities;

FIG. 76 illustrates a diagrammatical flowchart representing per presently-disclosed subject matter how to approach re-classifying where the classifier chooses the best parameter combination; and

FIG. 77 illustrates a diagrammatical flowchart representing per presently-disclosed subject matter the LAFR process coarsely grouped into seven stages: truncation, set formation (or clustering), quality checking, set selection 1, set selection 2, sample selection (or cherry-picking), and prediction.

Repeat use of reference characters in the present specification and figures is intended to represent the same or analogous features or elements or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to technology which is a generalized local adaptive fusion regression (LAFR) process for quantitative analysis of molecular based spectroscopic data. Local target-adaptive calibration and prediction allows for onsite analysis by handheld devices. In certain respects, various embodiments of presently disclosed subject matter relate to building simple machine learning models adapted to the deployment domain for quantitative chemical analysis.

Hand-held measurement (spectral) devices make onsite chemical analysis in the field possible. However, the full potential of hand-held devices is still undeveloped due to the absence of a robust real-time training/prediction regression process. Specifically, in order to obtain a quantitative prediction of a property needed for a target sample analyte from the deployment domain, such as the amount of pulp content in a tree for potential harvesting, the training set must match (span) the particular target sample in terms of all sample specific hidden effects (e.g., variances due to physicochemical and measurement effects). In other words, in order to obtain a quantitative prediction of a target sample analyte, the calibration set must properly span the target sample in terms of all hidden matrix effects, e.g., physicochemical properties, relative to both spectral measurements (X) and amounts of all spectral responding factors (Y).

Matching the training sample set to the new deployment domain variance is a common problem to all machine learning disciplines. Hidden effects typically vary from sample to sample making it difficult to match one training set to all possible onsite target samples thereby confounding onsite analysis by hand-held devices. One machine learning framework to rectify the situation is local modeling. Local modeling is a framework that produces calibration sets matched to target samples. However, local modeling requires mining a database composed of thousands of training samples with varying hidden effects for a unique training set specifically matched to each new target sample. In other words, local modeling requires mining a library composed of thousands of analyte reference spectra with a vast array of implicit matrix effects with the intent of identifying a unique calibration set specifically matrix matched to the particular target sample being predicted.

Rather than forming one local model, the presently disclosed approach, termed local adaptive fusion regression (LAFR), forms hundreds of linear local models from a library with each model representing a distinct combination of hidden matrix effects. The LAFR approach considers local modeling as a classification situation where a target sample is classified into the local model with the most similar matrix effects. In other words, target samples are classified to the best matched linear training set.

Developed for presently disclosed LAFR is a measure termed the indicator of system uniqueness (ISU) that is a hybrid fusion algorithm based on over a hundred similarity measures using a novel cross-modeling procedure, with X and Y matches, and each sample prediction amount receives a membership value relative to the predicting calibration set. Results are presented for multiple near infrared (NIR) spectral datasets including a difficult soil library with nearly 100,000 reference samples. All datasets demonstrate the suitability of LAFR for handheld spectral devices.

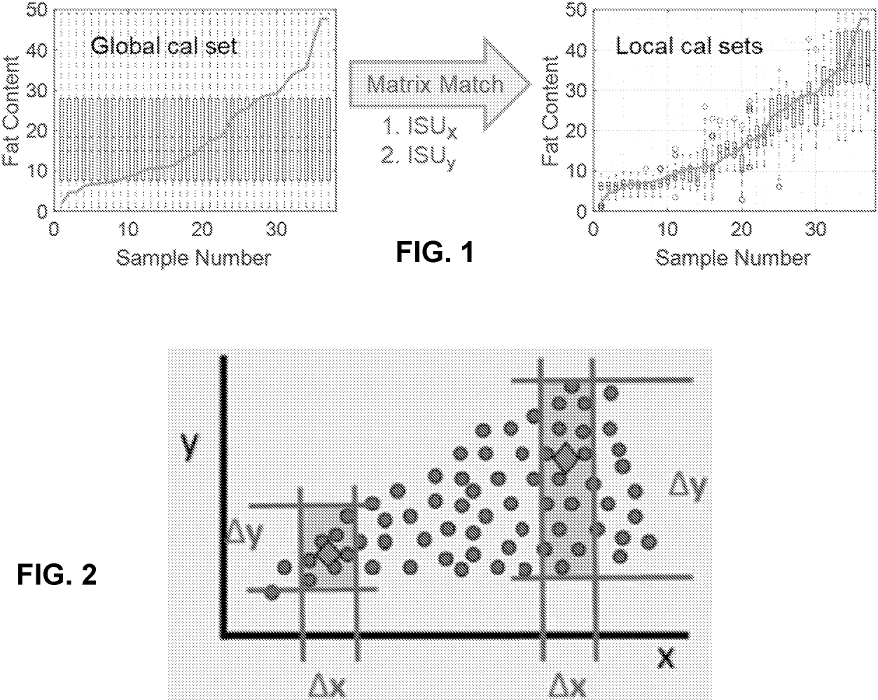

FIG. 1 illustrates an example of matrix matching from Global Calibration sets into Local calibration sets. Thus, FIG. 1 represents the presently disclosed approach resulting in of extracting matrix matched samples being extracted from large spectral libraries for local multivariate calibration.

An objective of the presently disclosed Local Adaptive Fusion Regression (LAFR) technology is to develop a local modeling method to form calibration sets from large or non-linear datasets. Such calibration sets (CalSets) should be matched to each target sample by both spectra and analyte amount. Data fusion is utilized to make robust each step of the process.

The following frames various concepts, beginning with Local Modeling. The theory behind the approach is to identify a calibration set from a greater library to increase prediction accuracy of a target sample. Common use cases include such as (1) Large datasets: local modeling mines through datasets of 1000+ samples to find the closest matched samples, and (2) Non-linear datasets: most non-linear data is sufficiently linear within a local range of a target point.

Examples of existing methods include:
CARNAC (Davies, A., et al.; Mikrochimica Acta, 1988 (1))
LOCAL (Shenk, A., Westerhaus, M.; J. Near Infrared Spectrosc. 1997 (5))
LWR (Naes, T., Isaksson, T., Kowalski, B.; Anal. Chem. 1990 (62))

There are shortcomings of standard methods. For example, most only use spectral similarity measures and cannot match the target’s analyte concentration precisely. FIG. 2 illustrates an example of a known data handling method, such as disclosed by Kalivas, J., Emerson, R.; Taking a big data approach to local spectral calibration. Chemometrics in Analytical Chemistry, 2016. Other shortcomings are that methods that match by analyte amount use a global model prediction and assume it to be fairly accurate. Also, standard methods generally rely on very few similarity measures (1-5 measures), which does not encompass enough similarity information.

Theory of the presently disclosed Data Fusion approach is that Data fusion combines multiple similarity metrics to obtain a holistic understanding of the similarity between samples. Local modeling is concerned with sample selection. Samples differ by spectral magnitude, shape, and analyte concentration. Samples selected need to be similar in all 3 categories. Many similarity measures are calculated, and fused for robusticity.

Capstones of presently disclosed approach include that it considers all possible user-defined variables. Local modeling depends on input hyperparameters (e.g. similarity merits, number of samples). Our presently disclosed approach considers all possible hyperparameter combinations (HPPCs), and selects the best overall calibration set (CalSet).

Other present capstones include utilizing Data Fusion. Fusion across many similarity merits robustifies the sample selection process. Also, for some embodiments of presently disclosed subject matter, match is based on analyte amount. Our presently disclosed matrix matching protocol harnesses the local regression vector to match target analyte magnitude.

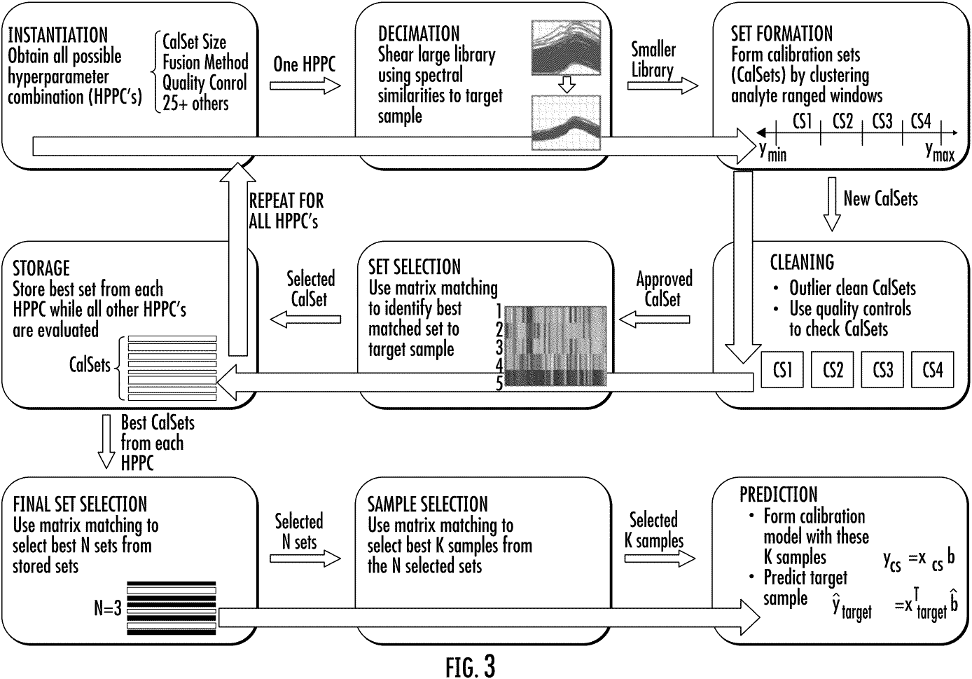

FIG. 3 illustrates an explained flowchart for the presently disclosed Local Adaptive Fusion Regression (LAFR) process for local modeling and calibration set selection.

Additional information which may be involved in presently disclosed embodiments relate to Hyperparameter Combinations (HPPCs). Nineteen user defined input hyperparameters define the LAFR protocol (CalSet size, quality control thresholds, etc.). LAFR determines all possible combinations of these 19, and determines the strongest CalSet defined by each. Further information can relate to Quality Controlling CalSets. CalSets should contain the predicted target analyte amount within their own distribution ( encapsulation), and CalSets should predict their own calibration samples well (e.g. strong R2, RMSEC, etc.). Still further information may relate to Matrix Matching. Twenty-nine similarity measures assess the degree of similarity between the target sample and each calibration set. “Cross-Modeling” is used to prevent chance matching.

The following relates to applicable performance metrics for consideration. Each target sample is predicted using the full LAFR protocol, and four metrics can be used to assess the overall prediction accuracy:
RMSEV: Root mean squared error of validation samples,
R2: Correlation of predicted values to truth,
Slope: Slope of regression line between prediction and truth, and
Intercept: Intercept of the same regression line.

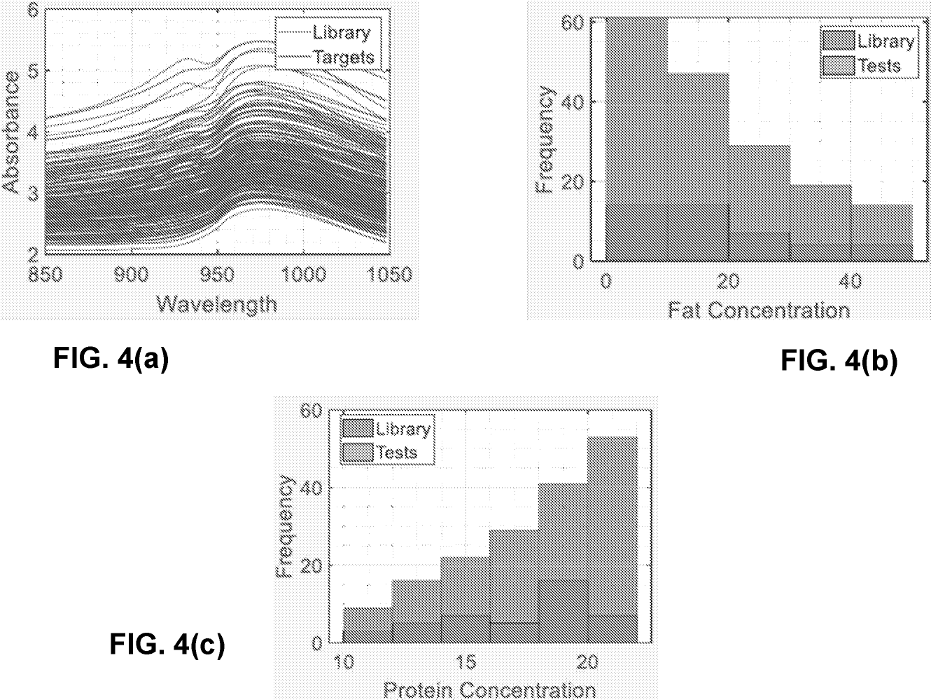

The following provides one dataset description example, involving Meat. Specifically, 170 samples of ground pork meat were used as the library to local model for 43 target samples. Spectra are measured in the near-infrared range with 100 wavelengths, with analytes protein, moisture, and fat. FIG. 4(a) illustrates spectra of library and target samples for Meat. FIGS. 4(b) and 4(c) illustrate histograms of Fat and Protein concentrations for the library and target samples, respectively. The utilized Library relates to Borggaard, C., Thodberg, H.H.; Optimal Minimal Neural Interpretation of Spectra. Analytical Chemistry, 1992.

Results for the Meat Fat, may be considered both by Global Model and Local Model data, as shown by the following Table 1:

TABLE 1

| | |
|---|---|
| Global Model | Local Model |
| R² = 0.976 | R² = 0.999 |
| Intercept = 0.365 | Intercept = -0.097 |
| Slope = 0.975 | Slope = 0.995 |
| RMSEV = 2.016 | RMSEV = 0.442 |

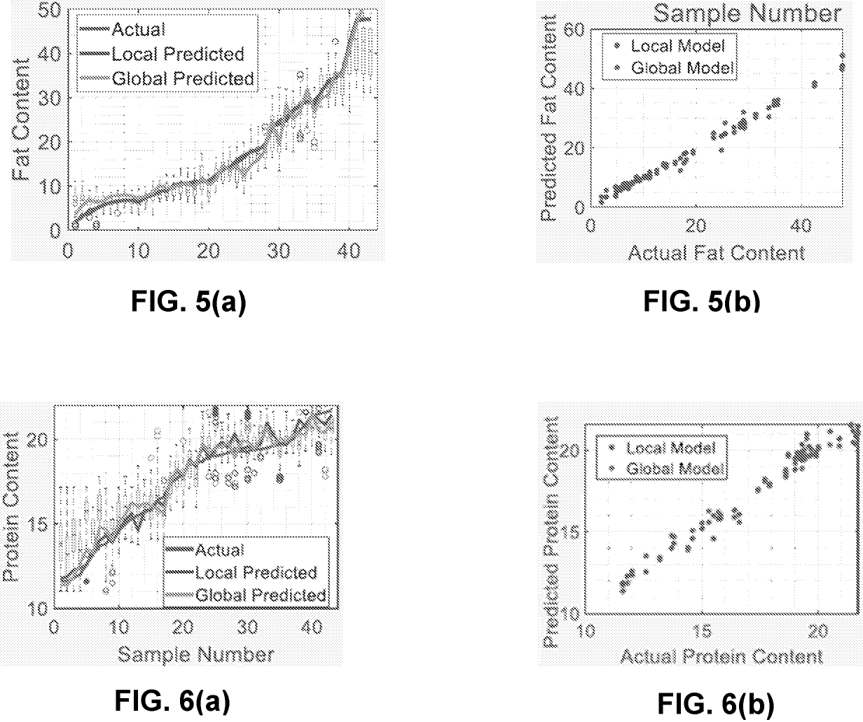

FIG. 5(a) graphically illustrates Global prediction and LAFR prediction of fat content for each sample, with the CalSet analyte distribution boxplots (upper). Actual and predicted fat content by the LAFR model and the global model for correlation analysis are shown by the graphical illustration of FIG. 5(b).

Results for the Meat Protein, may be considered both by Global Model and Local Model data, as shown by the following Table 2:

TABLE 2

| | |
|---|---|
| Global Model | Local Model |
| R² = 0.964 | R² = 0.978 |
| Intercept = 1.106 | Intercept = 0.191 |
| Slope = 0.947 | Slope = 0.998 |
| RMSEV = 0.597 | RMSEV = 0.474 |

FIG. 6(a) graphically illustrates Global prediction and LAFR prediction of meat protein content for each sample, with the CalSet analyte distribution boxplots (upper). Actual and predicted protein content by the LAFR model and the global model for correlation analysis are shown by the graphical illustration of FIG. 6(b). As shown by the example above, it may be concluded that LAFR is successful at identifying strong calibration sets matched by both analyte amount and spectra, that local modeling using non-linear libraries is strong using the LAFR protocol, that using data fusion and many hyperparameter combinations, LAFR becomes robust.

A similar dataset example involved ground pork meat. Specifically, 156 samples of ground pork meat were used as the library to local model for 37 target samples. Spectra from 850-1050 nm were measured in the near-infrared range with 100 wavelengths, again with analytes protein, moisture, and fat.

Further efforts discussed herein relate to analysis and optimization of LAFR to new datasets, particular interest in large datasets, and novel CalSet formation protocol. Part of the presently disclosed subject matter relates to the concept of what does it mean for samples to be similar. Subject matter relates to comprehensively characterizing similarity for spectral data. As a form of introduction, one may consider for example chemical data structure. In such context, objects may be regarded as being similar if many of their important properties are similar. From the perspective of spectroscopic samples, one may consider for example:
Analyte values: concentration of constituent of interest
Spectra: response fingerprint when externally stimulated
Matrix effects

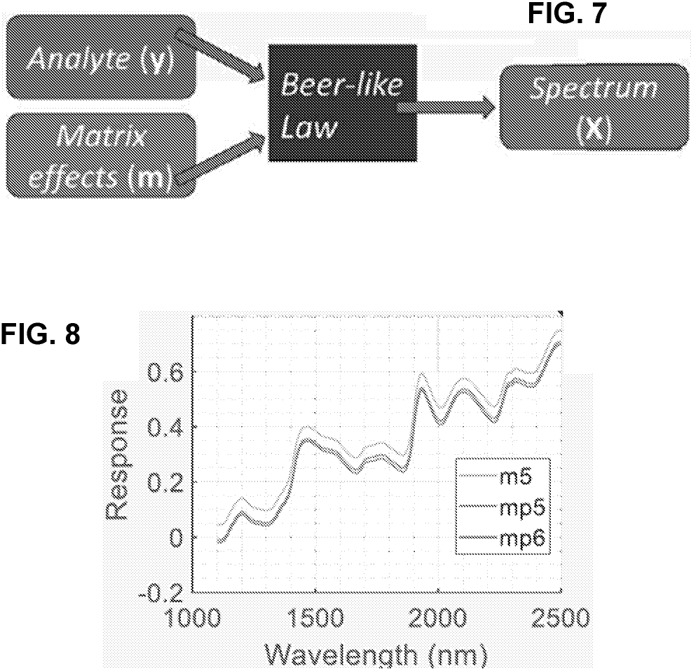

Matrix effects in this context relates to the confounding relationship between spectrum and analyte. Generally, sample conditions may relate to non-analyte chemical composition and all responding species. Measurement conditions may involve such as instrument novelties or baseline shift. FIG. 7 diagrammatically illustrates interplay of matrix effects.

Characterizing similarity generally may involve similarity assessment. The above three properties (analyte values, spectra, and matrix effects) are mutually dependent. If two are similar then all three are similar. Thus, it is straightforward to assess spectral similarity, such as Euclidean distance, Mahalanobis distance, Q-residual or other known approaches. It is more difficult to determine analyte or matrix effect similarity.

Similarity criterion characterizes the similarity between two samples to give a numerical indicator of similarity. Many applications need to quantify the similarity between samples, or between a sample and a calibration space.

Similarity applications may involve, for example, applicability domain/outlier detection. If target sample is similar to calibration samples then the model is likely applicable and accurate. Per calibration transfer, it can be possible to transfer a calibration from one set of matrix effects to encompass a different set. The degree of matrix effect dissimilarity determines which mechanism to use. Regarding matrix matching, one should preferably choose the best calibration set for each target sample. Per classification, one should preferably classify a target sample into the most similar class.

Generally speaking, the present objectives involve:
Holistically characterizing sample similarity,
Completing picture of spectral and analyte similarity,
Providing simple and robust for non-expert users,
Not requiring a-priori optimization knowledge, and
Integrating well with a wide variety of chemometric applications.

The following relates to various aspects of theory concerning presently disclosed subject matter. In particular, for assessing analyte similarity, a sample i with chemical composition y, matrix unaffected pure component spectra K, and matrix effects m is represented by:

$${\text{x}}_{i}^{T} = {\text{y}}_{i}^{T} \text{K} \text{+} {\text{m}}_{i}^{T} \qquad \text{(Eq. 1)}$$

Therefore for two samples i and j:

$$\begin{matrix}{\hat{y}}_{i} - {\hat{y}}_{j} = {\text{x}}_{i}^{T} \hat{\text{b}} \text{−} {\text{x}}_{j}^{T} \hat{\text{b}} \\ \text{=} \left({\text{y}}_{i}^{T} \text{K} \text{+} {\text{m}}_{i}^{T} - {\text{y}}_{j}^{T} \text{K} \text{−} {\text{m}}_{j}^{T}\right) \hat{\text{b}} \\ \text{=} \left({\text{y}}_{i}^{T} - {\text{y}}_{j}^{T}\right) \text{K} \hat{\text{b}} \text{+} \left({\text{m}}_{i}^{T} - {\text{m}}_{j}^{T}\right) \hat{\text{b}}\end{matrix} \qquad \text{(Eq. 2)}$$

The y factor is analyte matching while the m is matrix effect matching. Although confounded, there is still a rough assessment of analyte and matrix effect similarity. Generally, there can be preferably four requisite decisions to develop a similarity criterion:
1. Which similarity measures should be used?
   Many use only Mahalanobis distance and Q-residual
2. How to optimize similarity criteria?
   Many similarity measures require a tuning parameter (number of components)
3. Which method of comparing similarity?
   a) Calibration sample to target sample
   b) Calibration mean to target sample
4. How to fuse similarity scores?
   Many points of data must be combined into a value of overall similarity
   There are many options for this fusion protocol

Relating to the presently disclosed Indicator of System Uniqueness (ISU), an example of the four decisions to be made include as follows:
1. Which similarity measures should be used?
   26 spectral similarity merits
   18 analyte/matrix effect similarity merits
2. How to optimize similarity criteria?
   Principal components: fuse across windows and use a 99% variance rule
   Latent variables: Use a basic bias-variance trade-off
3. Which method of comparing similarity?
   Calibration sample and target sample compared to calibration mean, then differenced
4. How to fuse similarity scores?
   i. Average over tuning parameters and cal. samples
   ii. Use sum of ranking differences (SRD) to fuse similarity of each sample
   iii. Z-score the target sample SRD score with respect to the calibration samples scores (ISU_{X} and ISU_{y})
   iv. Determine Membership Factor (ISU_{xy}-MF) using z-tables.

The following is an example dataset involving corn.
80 corn samples measured on 3 different NIR (near-infrared spectroscopy) instruments
Analytes moisture, oil, protein, starch

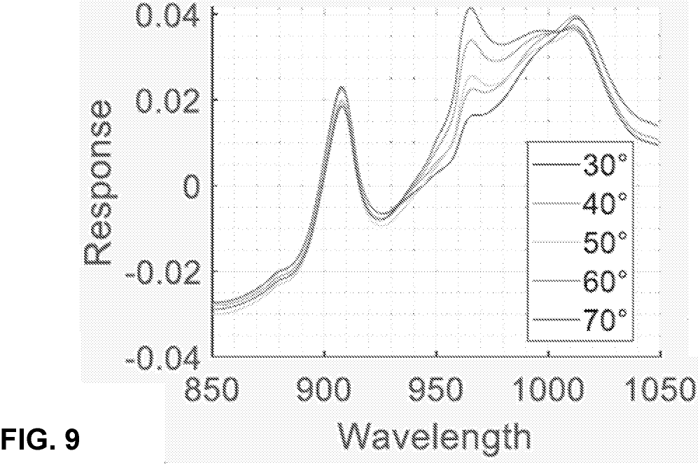

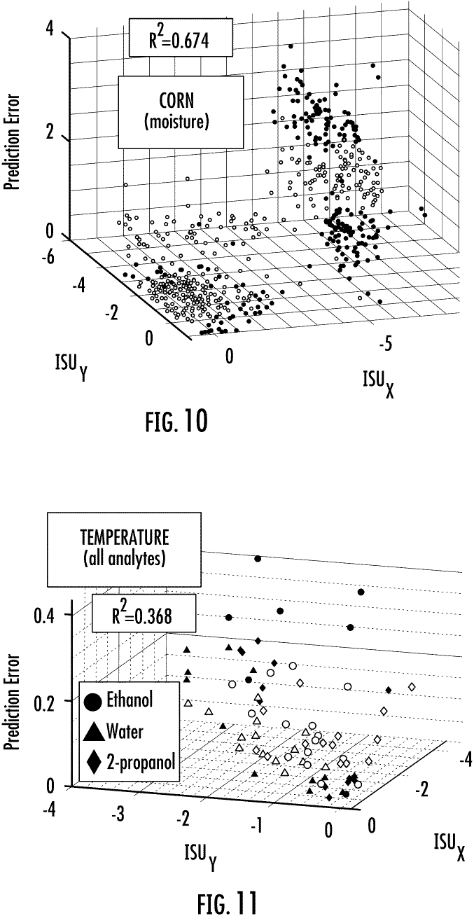

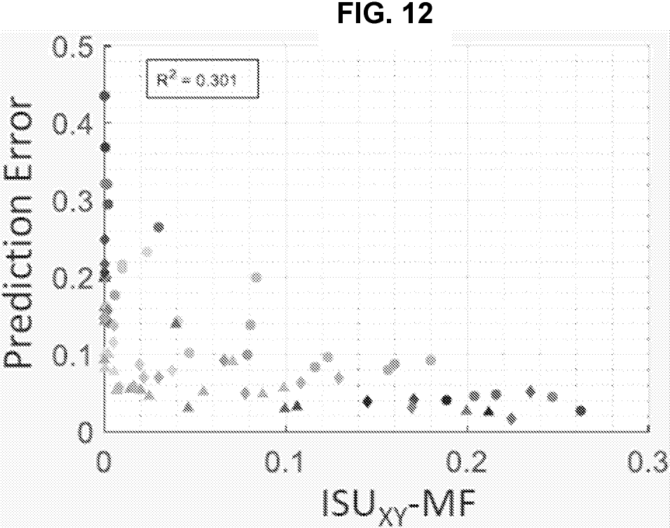

FIG. 8 graphically illustrates mean spectra of the corn samples from the three instruments. FIG. 9 graphically illustrates mean spectra of temperature samples from the five indicated temperatures, as follows: Temperature 16 ethanol/water/isopropanol mixtures measured at five temperatures (30, 40, 50, 60, 70 C).

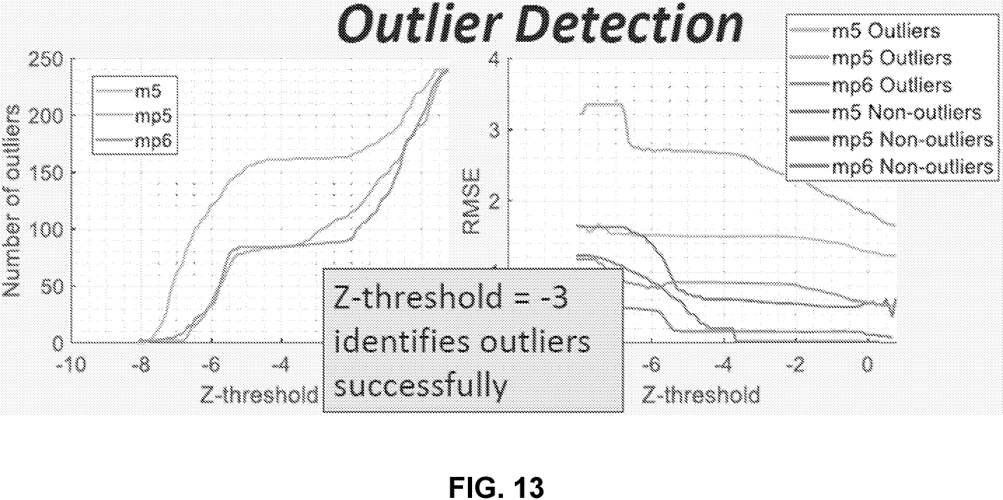

Results in the applicability domain show that dataset combinations with a low ISUₓ and ISU_{y} are predicted poorly. Per the following referenced illustrations, all samples from a source-target combination are of the same color/designation. FIG. 10 graphically illustrates prediction error of corn samples against their corresponding z-score ISUₓ and ISU_{y} results. Colors/designations correspond to the 9 possible source-target configurations. FIG. 11 graphically illustrates average prediction error of temperature datasets against average z-score ISUₓ and ISU_{y} results. Colors correspond to the 25 possible source-target configurations. Shapes are the three analytes, shown in the left-side of FIG. 11. FIG. 12 graphically illustrates average prediction error of temperature datasets against combined ISU_{xy} Membership Factor achieved by converting the ISUₓ and ISU_{y} to z-table probabilities, then multiplying the probabilities together. FIG. 13 relates to outlier detection, and graphically illustrates (on the left side thereof) the number of outliers against z-threshold for the three corn datasets as source, individually, and on the right-side thereof, illustrates RMSE (Root Mean Squared Error) of the samples identified as outliers or non-outliers from a particular source calibration set.

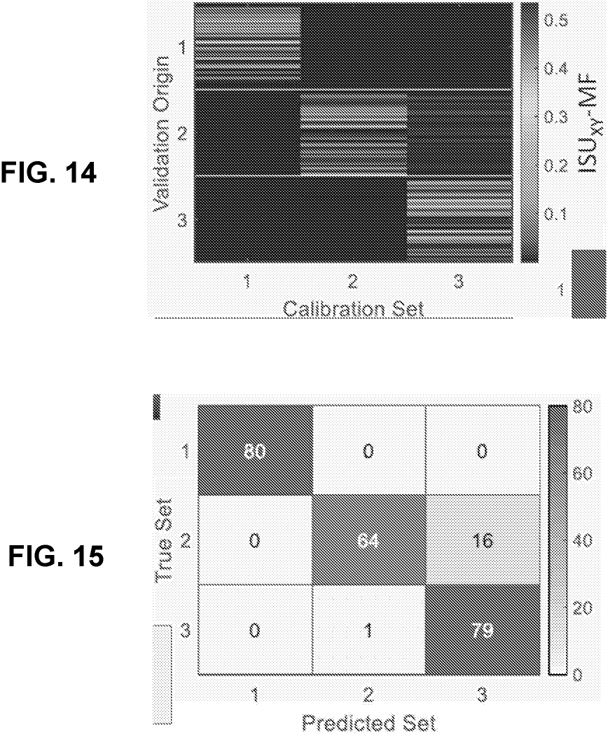

Regarding results in the context of matrix matching, CalSet-sample pairs with high ISU are well matrix matched to each other. FIG. 14 graphically illustrates 240 target samples from corn m5, mp5, and mp6, respectively, compared using the ISU to the three source calibration sets. FIG. 15 graphically illustrates a confusion matrix for matrix matching target samples into their actual calibration set of origin.

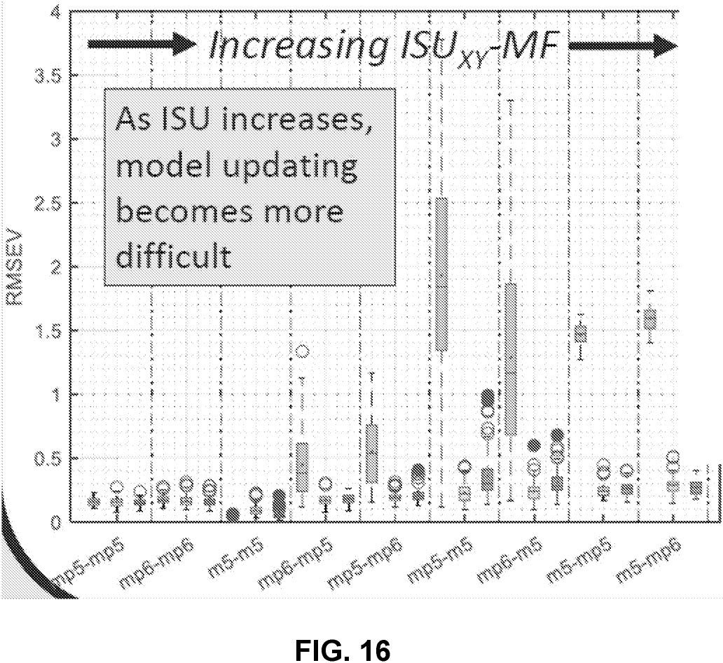

Regarding results in the context of calibration transfer, FIG. 16 graphically illustrates RMSEV (root mean squared error of validation samples) of model updating mechanisms for the source calibration set updating to target conditions. Pink (upper bars) represent basic calibration (primary predicting secondary). Green (data collections in locations 12, 15, 118, 21, 24, 26, & 27 from zero point) represents null augmented regression (NAR), and blue (remaining items) represents local mean centering (LMC). All three select models are based on using MDPS (Markov decision processes), and are sorted laterally according to ISU_{XY}-MF from data in FIG. 10.

Conclusions of the foregoing may be considered in the context of accomplishments. These may include sample similarity characterized comprehensively using many spectral and analyte similarity merits, an optimization scheme which is simple and user-invariant, and readily and easily applied to four multivariate spectral applications. Considered in the context of takeaways, it is observed for samples to be similar, they must be similar in all regards, thus similarity criteria must tackle this, and in applications regarding similarity, the ISU is a robust, user-friendly, and powerful tool that should be used. In particular, as disclosed herein, use of the ISU is pertinent for a novel local modeling scheme.

The following more particularly relates to aspects of automatic unlabeled target-adaptive spectral models with target prediction, including (1) Local Adaptive Fusion Regression (LAFR) and (2) Null Augmented Regression (NAR). The following equations may be pertinent to presently disclosed spectral multivariate calibration/prediction:

$$\text{y} \text{=} \text{Xb} \qquad \text{(Eq. 3)}$$

where
y = m x 1 vector of analyte reference values for m calibration samples
X = m x n matrix of spectra for n wavelengths
b = n x 1 regression (model) vector

$$\hat{\text{b}} \text{=} {\text{X}}^{+} \text{y} \qquad \text{(Eq. 4)}$$

Biased regression solutions such as PLS, PCR, Tikhonov regularization (TR) including RR, and others; require meta-parameter (tuning parameter) selection

$${\hat{y}}_{\text{new}} = {\text{x}}_{\text{new}}^{T} \hat{\text{b}} \qquad \text{(Eq. 5)}$$

In consideration of possible calibration problems, it is recognized herewith that new target samples are often outliers to calibration data. Calibration samples must span expected target variances and correlations (matrix effects). This includes consideration of physicochemical properties (joint action of both physical and chemical processes). Further to be considered are secondary analyte correlations, environment and instrument measurement conditions, along with the nature of the specific agriculture products (for example, food brands, species, geographical region, growing seasons), medical diagnostics (inclusive of subject physiochemical dependent properties), and possible other hidden variables.

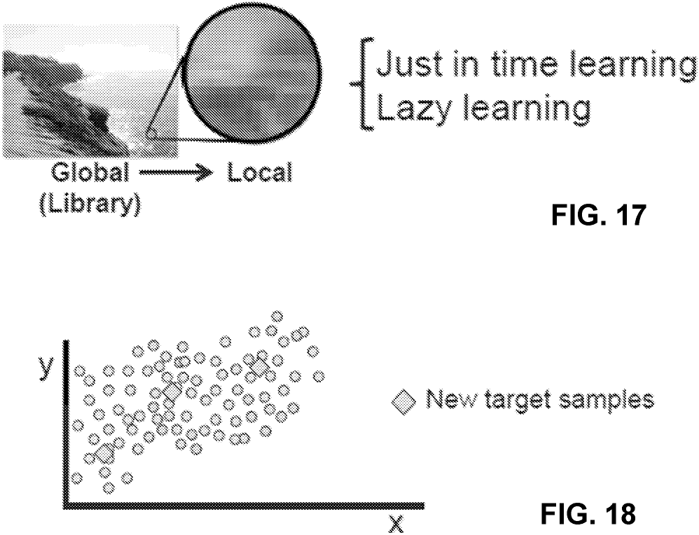

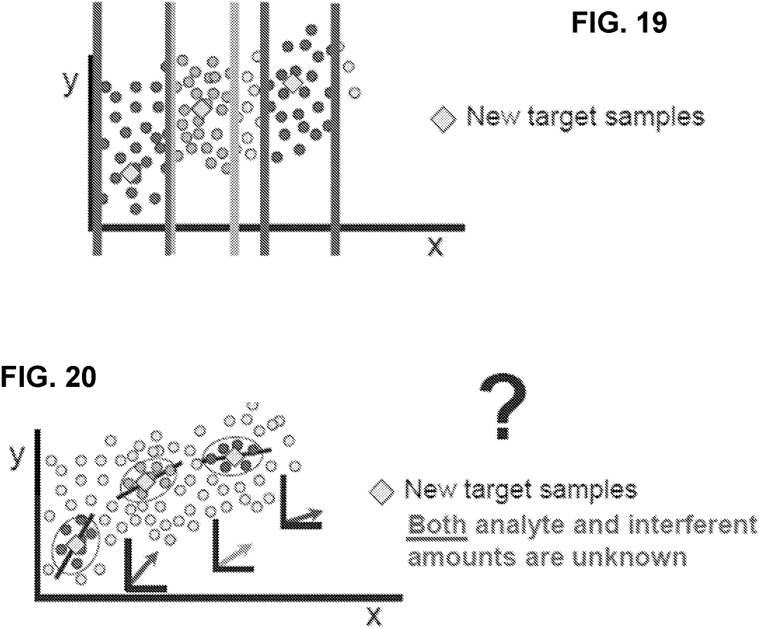

Relative to possible solutions, one (or a first solution) could be regarded as local modeling. As represented by FIG. 17, sometimes just in time learning or so-called lazy learning is sufficient to address going from a global library to a local condition. In some instances, the local calibration set may be a subset of library or historical data base. Target sample-wise models are preferably formed. Calibration set still needs to matrix match each target sample by functionality or operation as represented by the graphical illustrations of FIGS. 18, 19, and 20. In particular, FIG. 18 graphically represents new target samples among a library of historical data. FIG. 19 graphically represents that the calibration set needs to matrix match each target sample by X, hidden spectral effects. FIG. 20 graphically represents that the calibration set needs to matrix match each target sample by Y, analyte and unknown interferent amounts.

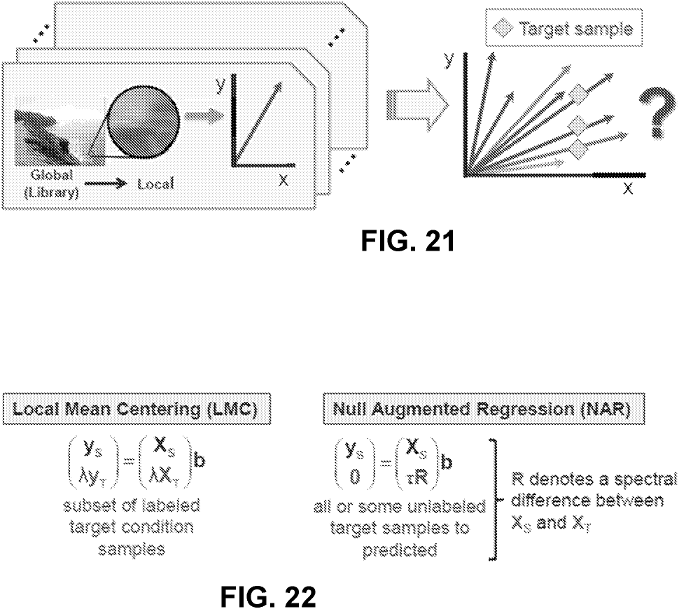

A second solution could be referred to as a bucket of models (or a form of ensemble learning approach). First, models may be from calibration sets with similar implicit matrix effects, as graphically represented by FIG. 21. Secondly, one may select a model (b̅) to predict the target sample, whereby model direction and magnitude characterizes calibration matrix effects.

A third solution may be referenced as model updating. One considers in particular combinations of (X_{source}, y_{source}) matrix effect differences relative to (X_{target}, y_{target}) matrix effects. From the perspective of domain adaptation, only X_{source} and x_{target} have shifted. Per a three-step approach, first retain all or part of historical primary source data, secondly modify model to predict target samples from new target conditions, and thirdly require model selection for multiple tuning parameters. FIG. 22 represents pertinent Local Mean Centering (LMC) and Null Augmented Regression (NAR) approaches.

Per presently disclosed Local Adaptive Fusion Regression (LAFR), one may combine solutions 1 and 2:
Per Local modeling, one would identify a calibration set matrix matched to target sample (x_{target} and ŷ_{target}) from analyte reference library;
As Adaptive to target sample implicit matrix effects, one would make calibration set changes if:
   1. analyte and/or interferent amounts shift
   2. different analyte for same sample (e.g., predicting clay, silt, sand, pH, etc. for one soil sample requires analyte specific calibration sets);
For Fusion of multiple X and analyte y similarity measures, one would identify a matrix matched calibration set (model); and then
Regression is used to form models.

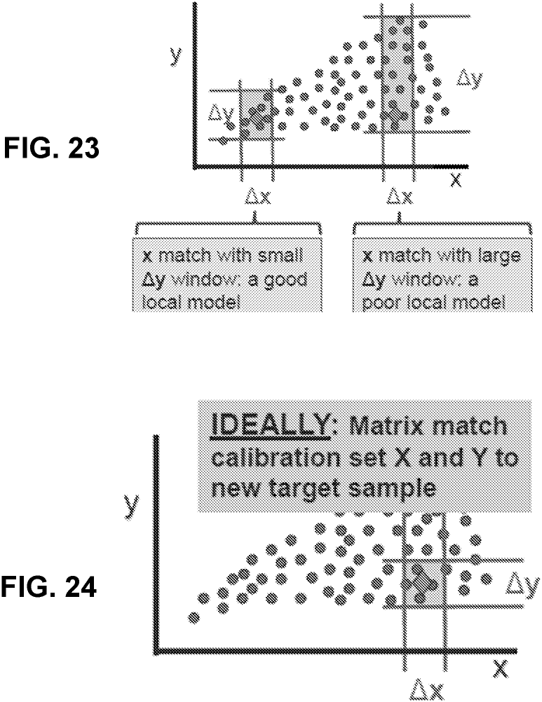

One problem is the need to target y information. “Localization should be carried out with respect to both spectrum and the analyte property” in accordance with Anderson RG, Osborne BG, Wesley IJ. J. Near Infrared Spectrosc. 11 (2003) 39-48, and Williams P. NIR News 30 (2019) 9-11. FIG. 23 graphically illustrates an example of a new sample (diamond). X matched does not mean y matched : 1. X matching selects same samples regardless of target Y_{analyte} amount or property; 2. Difficult to match target sample y_{analyte} and interferents since they are unknown; and 3. Calibration sample interferents are also unknown. Ideally, there is a matrix match calibration set X and y to new target sample, as graphically illustrated by FIG. 24.

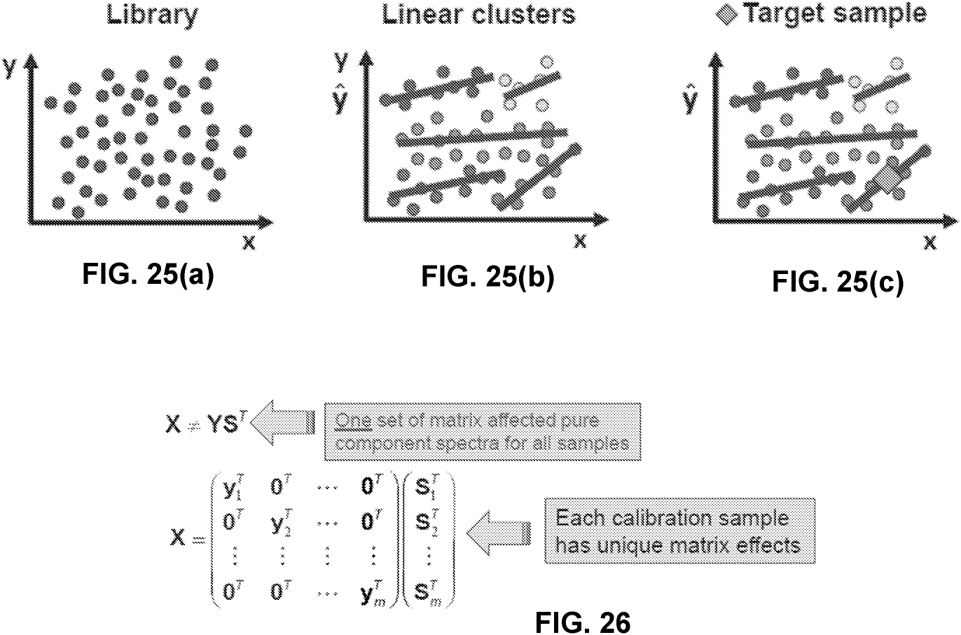

A macro overview of LAFR may be regarded as follows. Local modeling is treated as a classification problem. A target sample is classified by hidden matrix effects relative to each calibration set. In a first instance, the spectral library is broken into a collection of linear calibration sets, with sets that maintain similar implicit sample matrix effects identified by indicators of system uniqueness (ISU_{X} and ISU_{y}). Then, calibration sets are evaluated relative to target sample by ISUₓ and ISU_{y}, with the best ISU matched calibration model identified for final prediction. FIGS. 25(a), 25(b), and 25(c) graphically illustrate, respectively, examples of the initial library, then the determined linear clusters within the library, and the interplay with a target sample.

Decomposing a Sample Spectrum x, one may assume a linear Beer-Lambert law type relationship, which has a common representation:

$$\text{x} \text{=} {\text{y}}_{a} {\text{s}}_{a} + \sum {y}_{i} {\text{s}}_{i} + \text{e} \qquad \text{(Eq. 6)}$$

$$\text{x} \text{=} \text{Sy} \text{+} \text{e} \qquad \text{(Eq. 7)}$$

where yₐ, yᵢ = analyte and interference quantities in a sample,
sⱼ = matrix effected pure component spectrum at unit conc and pathlength corrected, and
e = random noise.

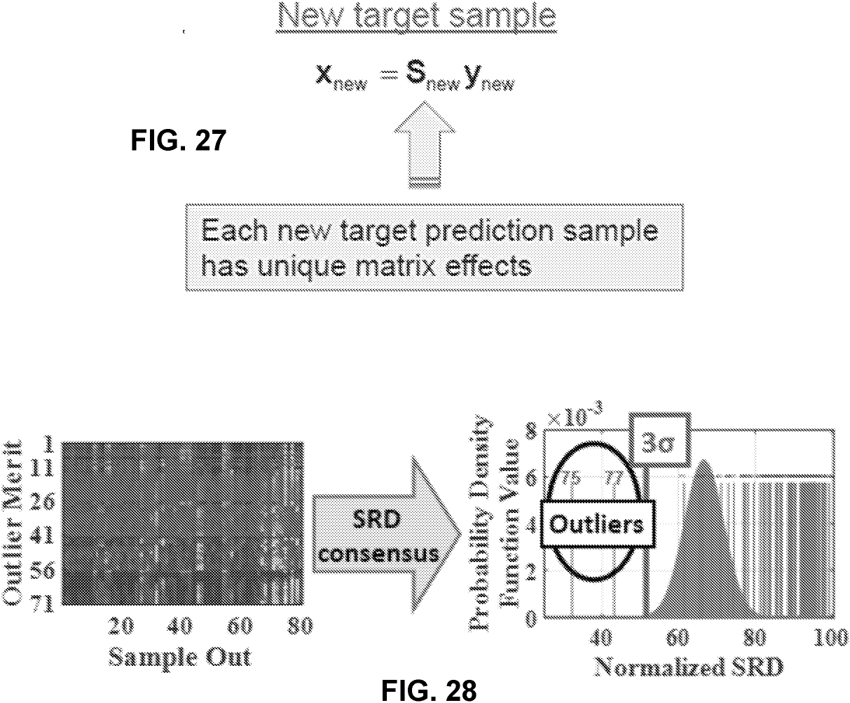

A calibration set (henceforth ignoring e, i.e. random noise) is represented by the mathematical representations of FIG. 26, where each calibration sample has unique matrix effects. FIG. 27 graphically represents treatment of a new target sample, where each new target prediction sample has unique matrix effects. In general, “hidden” S_{new} needs to be locally spanned by the calibration set S₁ ... Sₘ
1. Target sample x_{new} is spanned by calibration X: possibly a straightforward assessment;
2. Target sample y_{new} is spanned by calibration Y: possibly a tough assessment, y_{new} and Y are not known except for analyte values.

For consideration of further analysis of a sample spectrum:

$$\text{x} \text{=} {y}_{a} {\text{s}}_{a} + \sum {y}_{i} {\text{s}}_{i} \qquad \text{(Eq. 8)}$$

$${}_{\text{Let}}^{}{} {\text{S}}_{j} = {\text{PM}}_{j} {\text{ε}}_{i \text{then}} \text{x} \text{=} {y}_{a} {\text{PM}}_{a} {\text{ε}}_{a} + \sum {y}_{i} {\text{PM}}_{i} {\text{ε}}_{i} \qquad \text{(Eq. 9)}$$

Where ε = quantum mechanical matrix effect free spectrum (isolated molecule).

Further, for sample dependent terms (diagonal matrices):
P = pathlength dependent on wavelength relative to sample physical conditions
M = matrix effect dependent on:
   sample type: gas, liquid, mixture (solution or solid)
   molecular and ionic interactions
   concentrations relative to sample analyte and all interferent amounts
   environmental (measurement) and instrumental conditions
   wavelength

Then, for Eq. 8 and Eq. 9 above, the following holds:

$$\text{x} \text{=} {y}_{a} {\text{ε}}_{a} + \sum {y}_{i} {\text{ε}}_{i} + {\text{m}}_{t o t a l} = \text{Ey} \text{+} {\text{m}}_{t o t a l} \qquad \text{(Eq. 10)}$$

Where m = Pmₐ + ∑Pmᵢ and mₜₒₜₐₗ = catch all of any matrix effect altering a spectrum; instrument, measurement, chemical and physical interactions, etc.

When considering a calibration set:

$$\text{X} \text{=} {\text{YE}}^{T} + {\text{M}}_{t o t a l}^{T} \qquad \text{(Eq. 11)}$$

where Mₜₒₜₐₗ spans the calibration set of matrix effects.

For a specific calibration sample:

$$\begin{matrix}{\hat{y}}_{c a l} = {\text{x}}_{c a l}^{T} \hat{\text{b}} \\ \text{=} {y}_{a , c a l} {\text{ε}}_{a}^{T} \hat{\text{b}} \text{+} {\sum {y}_{i , c a l} {\text{ε}}_{i}^{T} \hat{\text{b}} \text{+} \text{m}}_{t o t a l , c a l}^{T} \hat{\text{b}}\end{matrix} \qquad \text{(Eq. 12)}$$

For a new target sample:

$$\begin{matrix}{\hat{y}}_{n e w} = {\text{x}}_{n e w}^{T} \hat{\text{b}} \\ \text{=} {y}_{a , n e w} {\text{ε}}_{a}^{T} \hat{\text{b}} \text{+} {\sum {y}_{i , n e w} {\text{ε}}_{i}^{T} \hat{\text{b}} \text{+} \text{m}}_{t o t a l , n e w}^{T} \hat{\text{b}}\end{matrix} \qquad \text{(Eq. 13)}$$

The matrix is matched when:

$$\begin{matrix}{y}_{a , c a l} {\text{ε}}_{a}^{T} \hat{\text{b}} \text{+} \sum {y}_{i , c a l} {\text{ε}}_{i}^{T} {\hat{\text{b}}}_{\text{+m}}^{t o t a l , c a l} \hat{\text{b}} \text{=} {y}_{a , n e w} {\text{ε}}_{a}^{T} \hat{\text{b}} \text{+} \\ \sum {y}_{i , n e w} {\text{ε}}_{i}^{T} \hat{\text{b}} \text{+} {\text{m}}_{t o t a l , n e w}^{T} \hat{\text{b}} \\ {}_{\text{Let}} {\text{ε}}_{a}^{T} \hat{\text{b}} \text{=} a , {\text{ε}}_{i}^{T} \hat{\text{b}} \text{=} i \\ {\text{y}}_{a , c a l} a + {\sum }_{{\text{y}}_{i , c a l} i \text{+} {m}_{t o t a l , c a l} \approx {\text{y}}_{a , n e w} a +} {\sum }_{{\text{y}}_{i , n e w} i + {m}_{t o t a l , n e w}}\end{matrix} \qquad \text{(Eq. 14)}$$

If m_{total,cal} ≈ m_{total,new}, y_{a,cal} ≈ y_{a,new}, y_{i,cal} ≈ y_{i,new}, then the two samples are matrix matched and ŷ_{a,cal} ≈ ŷ_{a,new}

If ŷ_{a,cal} ≈ ŷ_{a,new} for the calibration set, then:
1.Calibration samples have M_{cal} matrix matched to m_{new}
2.Calibration samples have Y_{cal} matrix matched to y_{new}

As to potential problems with ŷ_{a,cal} ≈ ŷ_{a,new}, predictions for one calibration sample can be similar but not matrix matched samples. One can obtain chance prediction equivalency, as follows:

$$\begin{matrix}{\text{y}}_{a , c a l} {\text{ε}}_{a}^{T} \hat{\text{b}} \text{+} \sum {\text{y}}_{i , c a l} {\text{ε}}_{i}^{T} {\hat{\text{b}}}_{\text{+m}}^{t o t a l , c a l} \hat{\text{b}} \text{=} {\text{y}}_{a , n e w} {\text{ε}}_{a}^{T} \hat{\text{b}} \text{+} \\ \sum {\text{y}}_{i , n e w} {\text{ε}}_{i}^{T} \hat{\text{b}} \text{+} {\text{m}}_{t o t a l , n e w}^{T} \hat{\text{b}} \\ {\text{y}}_{a , c a l} a + \sum {\text{y}}_{i , c a l} i + {m}_{t o t a l , c a l} = {\text{y}}_{a , n e w} a + \sum {\text{y}}_{i , n e w} i + {m}_{t o t a l , n e w} \\ {\hat{\text{y}}}_{a , c a l} \approx {\hat{\text{y}}}_{a , n e w}\end{matrix} \qquad \text{(Eq. 15)}$$

Linear combinations of different concentration and matrix effect values can produce ŷ_{a,cal} ≈ ŷ_{a,new} . Confounding matrix effects which can occur include many combinations give similar x, and similar x’s does not mean similar matrix effects. Chance prediction equivalency reduces with multiple calibration samples. Not all samples in a calibration set can have chance prediction equivalency. Presently disclosed LAFR uses a novel cross-modeling to further reduce chance prediction equivalency.

The following concerns presently disclosed Indicators of System Uniqueness (ISUₓ). ISUₓ in present context is a holistic characterization of implicit X sample-wise differences between target and calibration sample matrix effects. Values do depend on sample y values (x = Ey + m). Comparing 15 similarity measures may be represented:

$$- {\text{x}}_{n e w} ⇔ {\text{x}}_{c a {l}_{i}} \text{and} \left({\text{x}}_{n e w} {\text{x}}_{n e w}^{T}\right) ⇔ \left({\text{x}}_{c a {l}_{i}} {\text{x}}_{c a {l}_{i}}^{T}\right) \qquad \text{(Eq. 16)}$$

Some are based on SVD singular vectors (PCs), with Windows of PCs used to eliminate optimization, with for example Mahalanobis distance: all distances used from 1 PC up through the 99% rule, or with 8 similarity measures using PC windows.

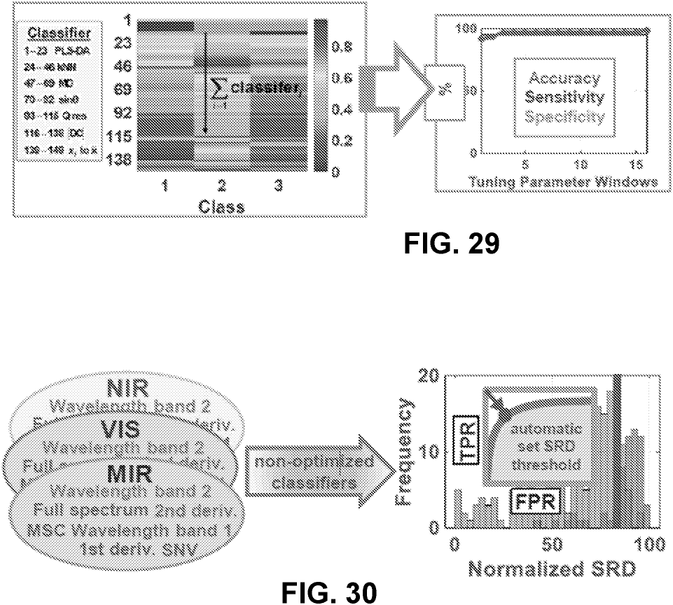

The following relates to applications of X matching with Windows (ISU_{X}). For example, outlier detection without tuning parameter selection is represented by the graphical illustration of FIG. 28 (using figure source DOI: 10.1021/acs.analchem.7b00637 Anal. Chem. 2018, 89, 5087-5094). Consensus classification using non-optimized classifiers is represented by FIG. 29 (using figure source DOI: 10.1021/acs.analchem.7b04399 Anal. Chem. 2018, 90, 4429-4437). Self-optimized one-class classification (class modeling) is represented by FIG. 30 (using figure source DOI: 10.1021/acs.analchem.7b00017 Anal. Chem. 2020, 92, 5354-5361).

The following relates to Indicators of System Uniqueness (ISU_{y}). ISU_{y} in present context is holistic characterization of implicit y sample-wise differences between target and calibration sample matrix effects, where values do depend on sample X values (x = Ey + m). Measures involve the interaction of

$$\overline{\text{b}}$$

with X and x_{new}, where

$$\hat{\text{b}}$$

has magnitude and direction and

$$\hat{\text{b}}$$

carries sensitivity, selectivity, net analyte single (NAS) information. 17 similarity measures compare variations of prediction value:

$$- {\hat{y}}_{n e w} ⇔ {\hat{y}}_{c a l} \qquad \text{(Eq. 17)}$$

Models are automatically selected using U-curves.

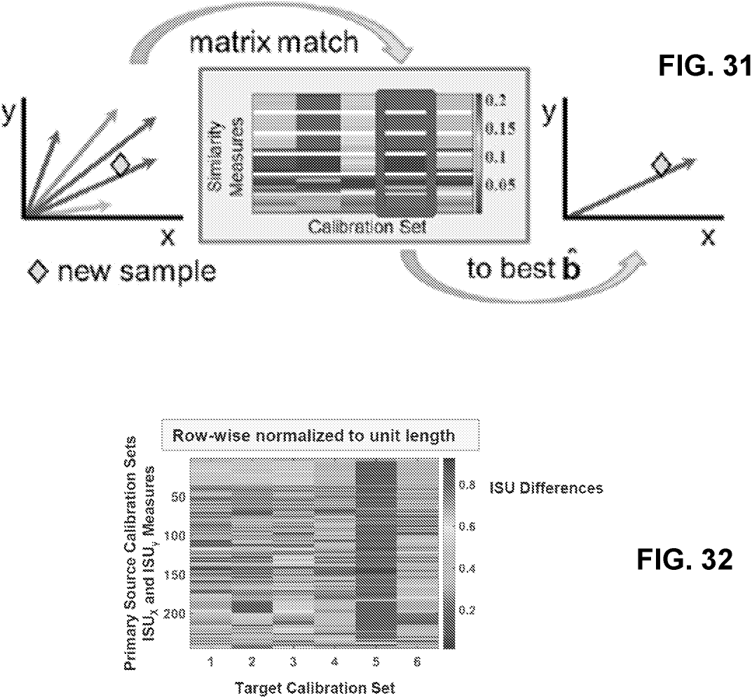

FIG. 31 graphically represents leveraging model regression vectors for matrix matching (using figure source DOI: 10.1021/acs.analchem.9b03302 Anal. Chem. 2020, 92, 815-823).

The following relates to cross-modeling for robust matrix matching, including description of Self- and Cross-model calibration sets with the new target sample. For self-model differences: 1. For a calibration set, remove a calibration sample and form a model with remaining samples; 2. Compute ISUₓ and ISU_{y} for the target and removed calibration samples relative to the remaining calibration samples and model; 3. Replace the calibration sample and repeat 1-3; 4. Compute the sum of mean ISU differences between the target and calibration samples as follows:

$$\left({\sum }_{i = 1}^{m} \left|{\text{ISU}}_{{\text{x}}_{i}} - {\text{ISU}}_{\text{x} n e {w}_{i}}\right| / m\right) + \left({\sum }_{i = 1}^{m} \left|{\text{ISU}}_{{\text{y}}_{i}} - {\text{ISU}}_{\text{y} n e {w}_{i}}\right| / m\right) \qquad \text{(Eq. 18)}$$

; and 5. Repeat 1-4 for each calibration set.

For cross-model differences: 6. Use a calibration sample set and its model as the primary source to compute ISUₓ and ISU_{y} for each other calibration sample set and new sample as the target samples; 7. Compute the sum of mean ISU differences between respective target calibration set samples and the new target sample; and 8. Repeat 6 and 7 with each calibration set acting as the primary source calibration set.

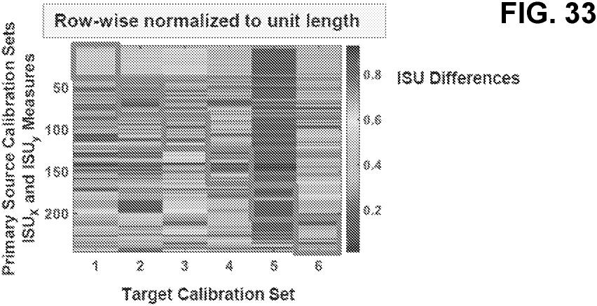

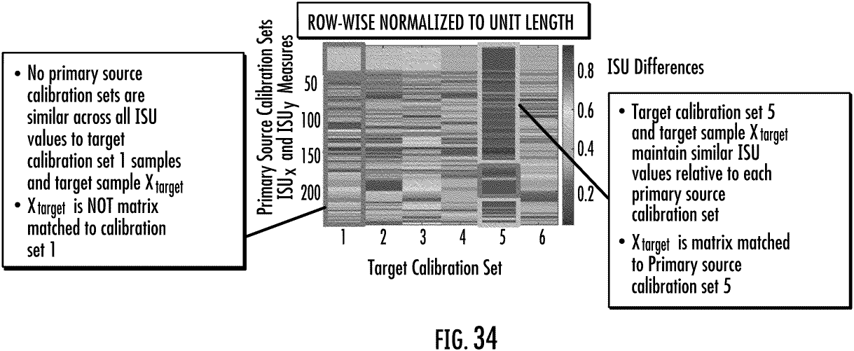

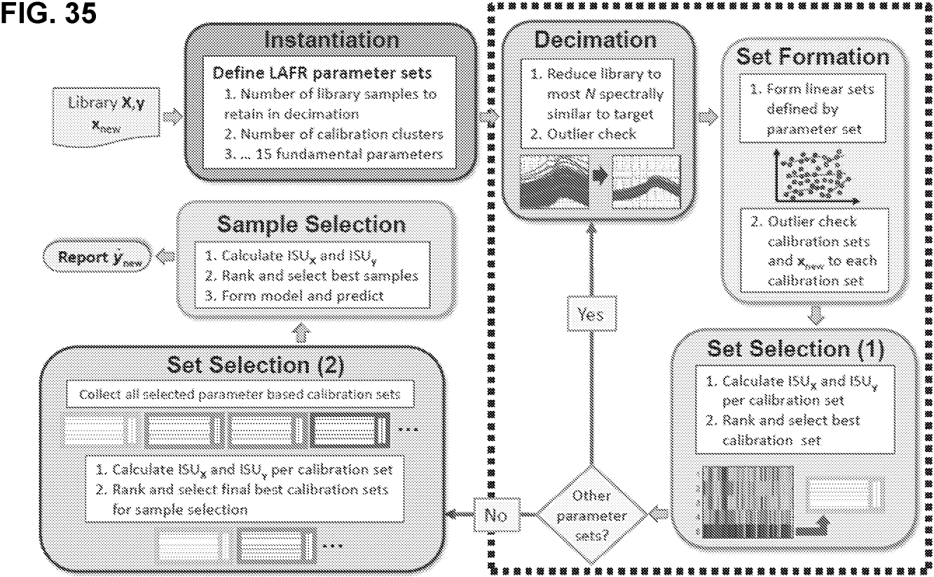

The following relates to best matrix matched calibration set as having minimum self- and cross-modeling ISU differences. For Self- and Cross-Model ISU differences, reference is made to the mean of ISU differences across 6 calibration sets, as graphically represented by FIG. 32. Diagonal blocks are self-model ISU differences, where x_{new} is matrix matched to primary source calibration set 5, per the graphic representation of FIG. 33. Off-diagonal blocks are cross-model ISU differences, as represented by FIG. 34. Different ISU measures indicate x_{target} is matrix matched to all calibration sets. Fusion of ISU measures per column identifies set 5 as best, in this example.

FIG. 35 illustrates in block diagram format a flowchart for an overview of the presently disclosed Local Adaptive Fusion Regression (LAFR) process subject matter. Fundamental Parameter Sets comprise overall considerations, Decimation, Outlier Cleaning, Set Formation, Matrix Matching, and Set and sample selection.

The overall parameter sets may include the number of PCs and the Partial Least Squares (PLS) model selection. The Decimation facets involve spectral similarity measures and the number of samples post-decimation. Outlier cleaning relates to outlier detection measures, the number of outliers to remove each iteration, whether to remove when checking unlabeled x_{target}, and self-prediction thresholds.

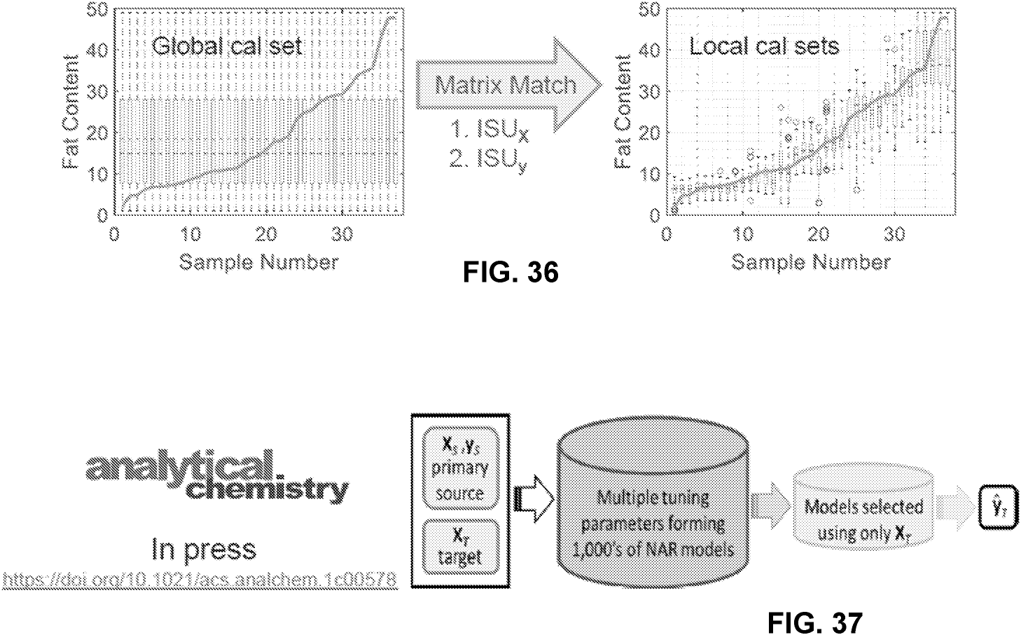

The subject set formation involves the number of calibration sets, the first cluster sample and initial distance measure, and ISU_{X}/ISU_{y}/E weighting on set formation. Present matrix matching involves similarity measures and use of the fusion rule. Set and sample selection involves N CSWiM sets and K SWiM samples. FIG. 36 graphically represents tracking presently disclosed matrix matching functionality going from Global cal set to Local cal sets, in a “fat content” example.

A typical or exemplary parameter set for soil may include:
SRD Filtering: {Nearest 500 samples}, {if small dataset, no SRD filtering}
Outlier SRD Threshold: -3 SDs
Clustering ISU_{X}/ISU_{y}/E weights: {1/2/1}, {1/2/4}, {1/1/4}
Number of Cal Sets: {6}, {8}, {10}
Self Prediction Threshold: {0.8 R²}, ŷ_{cal,min} ≤ ŷ_{target} ≤ ŷ_{cal,max}
Final sets before SWiM: N=5
Number of SWiM Samples: K=100 (depends on library size)

One exemplary summary of unique components of presently disclosed LAFR may be listed as follows:
1. LAFR forms models by an understandable process • Based on fundamental physicochemical properties and principles with a Beer’s law-like relationship
2. Final LAFR calibration sets bracket target sample analyte values
3. Adjustable parameters are self-optimizing
4. Prediction reliability by probability of target sample membership in final calibration (ongoing)

A further presently disclosed approach may be referred to as Solution 3: Model Updating. For such approach, in conjunction with domain adaptation: only X_{source} and X_{target} have shifted. This results in retaining all or part of historical primary source data, and modifying model to predict target samples from new target conditions, as represented in part by Table 3 below.

TABLE 3

| | |
|---|---|
| Local Mean Centering (LMC) | Null Augmented Regression (NAR) |
| $\left(\begin{matrix}{\text{y}}_{\text{s}} \\ \text{λ} {\text{y}}_{\text{T}}\end{matrix}\right) = \left(\begin{matrix}{\text{X}}_{\text{s}} \\ \text{λ} {\text{X}}_{\text{T}}\end{matrix}\right) \text{b}$ y_{T} and X_{T} subset of labeled target condition samples | $\left(\begin{matrix}\left(\begin{matrix}{\text{y}}_{\text{s}} \\ 0\end{matrix}\right) = \left(\begin{matrix}{\text{X}}_{\text{s}} \\ \text{tR}\end{matrix}\right) \text{b} \\ \text{R} \text{all of some unlabeled target} \\ \text{samples to be predicted}\end{matrix}\right\} \text{R denotes a spectral} {\text{difference between X}}_{\text{s}} {\text{and X}}_{\text{T}}$ |

[symbol]

[symbol]

indicates text missing or illegible when filed

The presently disclosed process requires model selection for multiple tuning parameters, as represented for example by the block diagram illustrations of FIG. 37. Some possibilities are represented by Table 4 below, re all models obtained by Partial Least Squares (PLS) model selection.

TABLE 4

| | |
|---|---|
| $\left(\begin{matrix}{y}_{\text{S}} \\ τ {y}_{\text{T}}\end{matrix}\right) = \left(\begin{matrix}{\text{X}}_{\text{S}} \\ τ {\text{X}}_{\text{T}}\end{matrix}\right) \text{b}$ | min(∥X_{S}b - y_{S}∥+ r² ∥X_{T}b - y_{T}∥) (2 tuning parameters) |
| $\left(\begin{matrix}{y}_{\text{S}} \\ 0\end{matrix}\right) = \left(\begin{matrix}{\text{X}}_{\text{S}} \\ λ {\text{R}}_{y}\end{matrix}\right) b$ | |
| $\begin{matrix}{\text{R}}_{3} = \left({\text{μ}}_{\text{S}} - {\text{μ}}_{\text{TU}}\right) \\ {\text{R}}_{2} = \left(\frac{1}{{\text{m}}_{\text{s}}} {\text{X}}_{\text{S}}^{\text{T}} {\text{X}}_{\text{S}} - \frac{1}{{\text{m}}_{\text{TU}}} {\text{X}}_{\text{TU}}^{\text{T}} {\text{X}}_{\text{TU}}\right)\end{matrix}$ | min(∥X_{S},b -y_{S}∥+ λ³ ∥R₂b∥) (2 tuning parameters) |
| $\left(\begin{matrix}{y}_{\text{S}} \\ 0 \\ 0\end{matrix}\right) = \left(\begin{matrix}{\text{X}}_{\text{S}} \\ λ {\text{R}}_{\text{3}} \\ η {\text{R}}_{2}\end{matrix}\right) \text{b}$ | min(∥X_{S}b-y_{S}∥+ λ²∥R₁b∥+ η²∥R₂b∥) (3 tuning parameters) |
| $\left(\begin{matrix}{\text{y}}_{\text{S}} \\ 0 \\ r {y}_{\text{T}}\end{matrix}\right) = \left(\begin{matrix}{\text{X}}_{\text{S}} \\ λ {\text{R}}_{r} \\ r {\text{X}}_{\text{T}}\end{matrix}\right) \text{b}$ | min(∥X_{S}b-y_{S}∥+ λ²∥R₁b∥+ τ²∥X_{T} b-y_{τ}∥) (3 tuning parameters) |

1 - Andries E, Kalivas JH, Gurung A. J. Chemom. 33 (2018) 1-20
2 - Nikzad-Langerodi R., Zellinger W, Lughofer E, Saminger-Platz S. Anal. Chem. 90 (2018) 6693-6701.

For model selection by MDPS (Markov decision processes), model diversity is shown in part by cosine of the angle between two models, as represented by Eq. 19:

$$cos {\left(θ\right)}_{i , j} = \frac{{\left({\hat{\text{b}}}_{i}\right)}^{T} \left({\hat{\text{b}}}_{j}\right)}{\left‖{\hat{\text{b}}}_{i}\right‖ \left‖{\hat{\text{b}}}_{j}\right‖} \qquad \text{(Eq.19)}$$

Models in the range 0.3 ≤ cos(θ) ≤ 0.5 are retained. Regarding prediction similarity, there are unlabeled prediction differences, and use of an overfitting safeguard (mean model 2-norm), per Eq. 20:

$$\begin{matrix}U P {D}_{i , j} = {\sum }_{n = 1}^{m} \left|{\text{x}}_{n}^{\text{T}} {\hat{\text{b}}}_{i} - {\text{x}}_{n}^{\text{T}} {\hat{\text{b}}}_{j}\right| \\ {\overline{\left‖\hat{\text{b}}\right‖}}_{i , j} = \frac{1}{2} \left(\left‖{\hat{\text{b}}}_{i}\right‖ + \left‖{\hat{\text{b}}}_{j}\right‖\right) \\ {\overline{R M S E C}}_{i , j} = \frac{1}{2} \left(R M S E {C}_{i} + R M S E {C}_{j}\right)\end{matrix} \qquad \text{(Eq. 20)}$$

An underfitting safeguard is mean source RMSEC as a calibration source. For final PS, for superscript RS, there are range scaled values, and ω weights the U-curve (0.4 is used), per Eq. 21:

$$P {S}_{i , j} = U P {D}_{i , j}^{R S} + ω \left({\overline{\left‖\hat{\text{b}}\right‖}}_{i , j}^{R S} + {\overline{R M S E C}}_{i , j}^{R S}\right) \qquad \text{(Eq. 21)}$$

FIG. 38 represents selecting models biased to unlabeled target samples.

In the exemplary case of soy seed samples, the following may be applicable:
NIR spectra on instruments R1 and R2 from 1100-2500 nm (300 wavelengths)
Moisture (5.9-18.4%), oil (29.0-43.4%), and protein (14.7-22.9%)
60 samples: 100 data splits with 30 primary source; 15 target; and 3 for LMC references
Updating from R1 (primary source) to R2 (target)

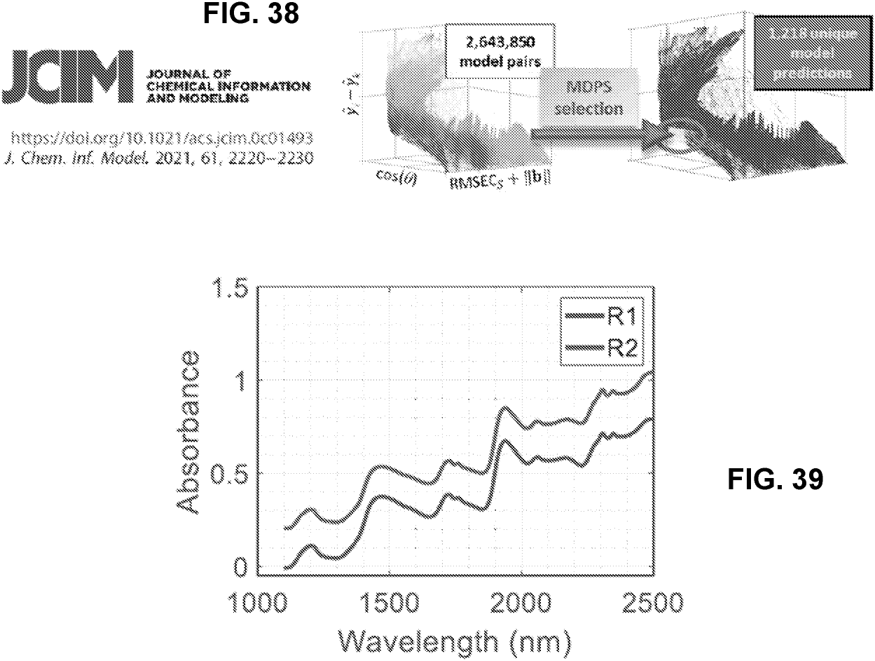

The overall goal is to form and select NARE models without labels that perform equivalently to LMC models with labels. FIG. 39 graphically illustrates R1 to R2 results, of moisture re soy seed samples, and uses as background resource Bouveresse E, Hartmann C, Massart DL, Last IR, Prebble P A. Anal. Chem. 68 (1996) 982-990.

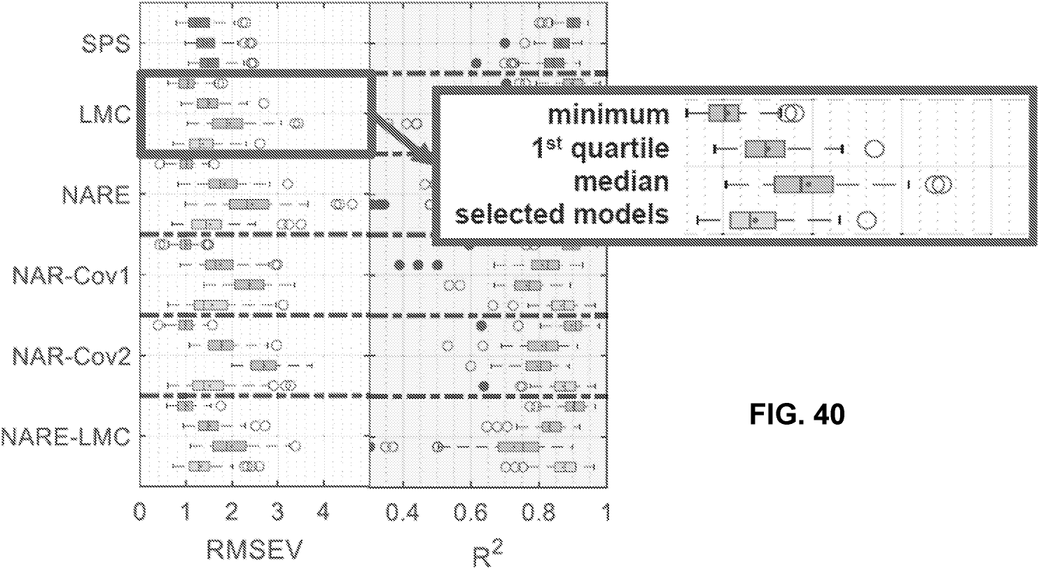

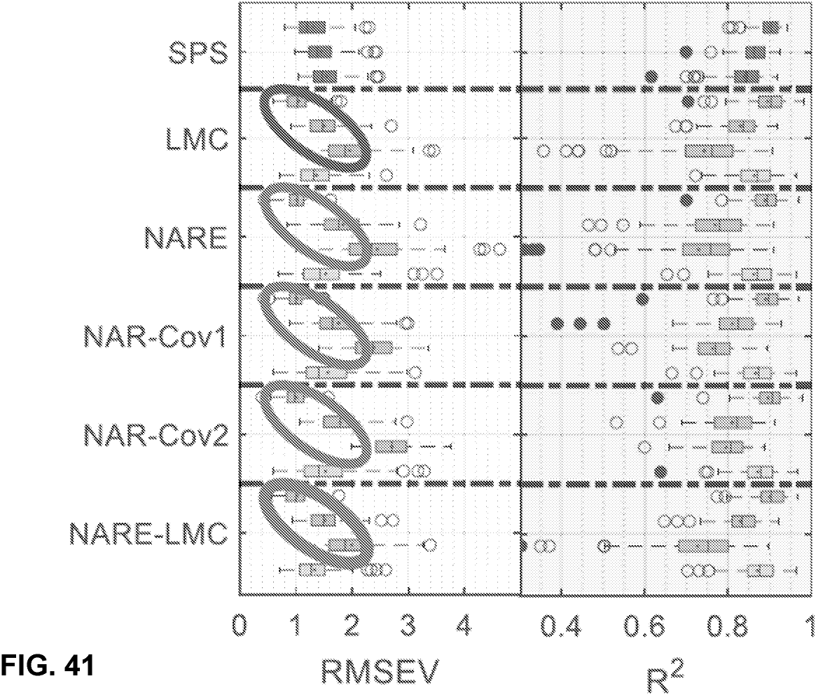

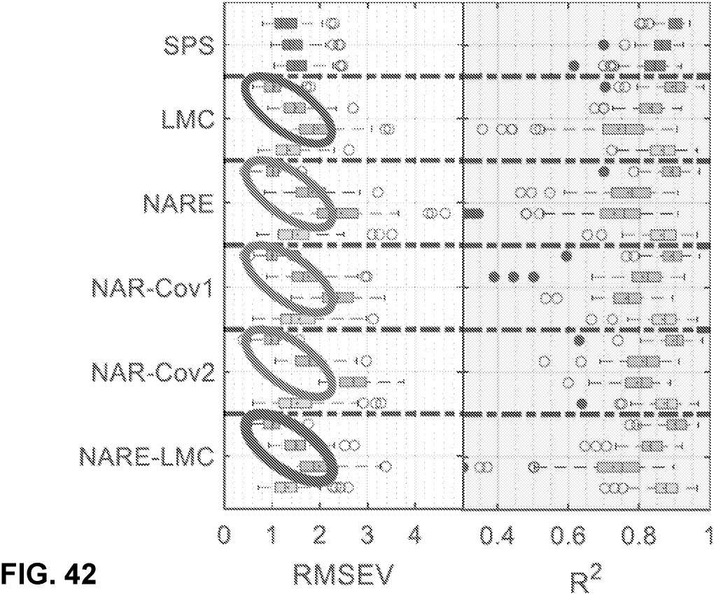

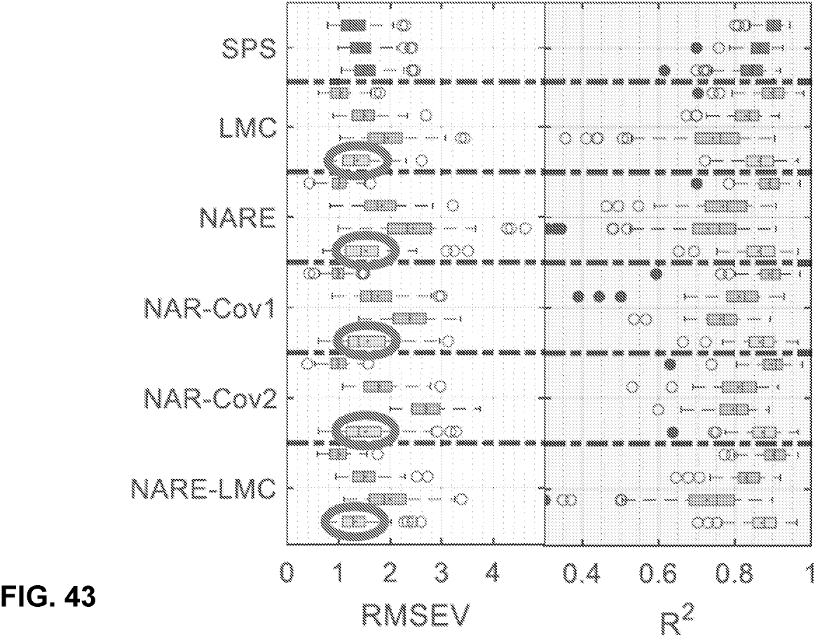

In conjunction with SPS (Secondary predicting secondary), FIG. 40 graphically illustrates target calibration predicting unlabeled target samples. All null augmented regression (NAR) approaches work equivalently to local mean centering (LMC) at minimum and 1st quartile root mean squared error of validation (RMSEV) (R²), as graphically represented by FIG. 41. Preferably, one may include a few target references to improve NAR at 1^{st} quartile and median, as graphically illustrated by FIG. 42. MDPS selects models at or below 1st quartile, with target samples used: (1) in MDPS to select NAR and LMC models, and (2) in R to form NAR models, as graphically represented in FIG. 43.

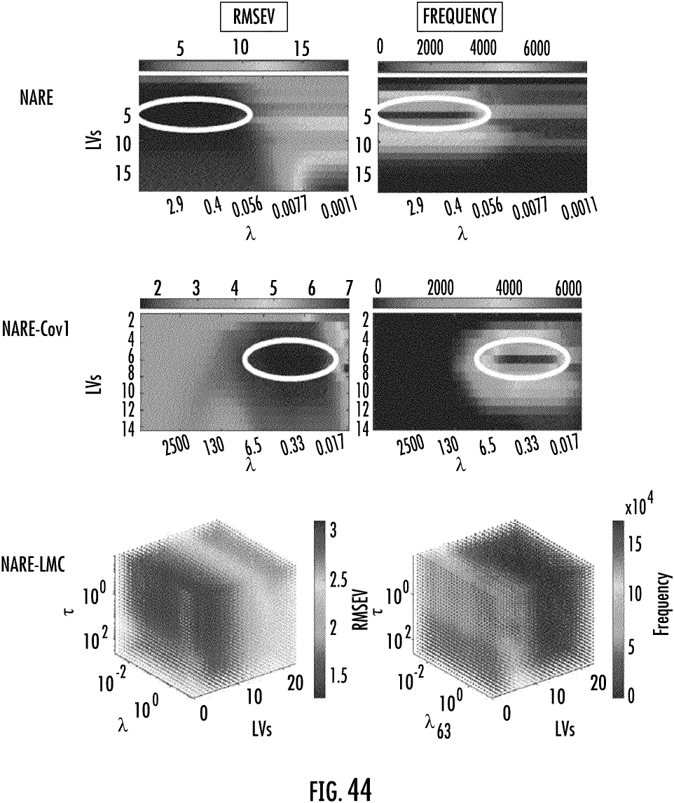

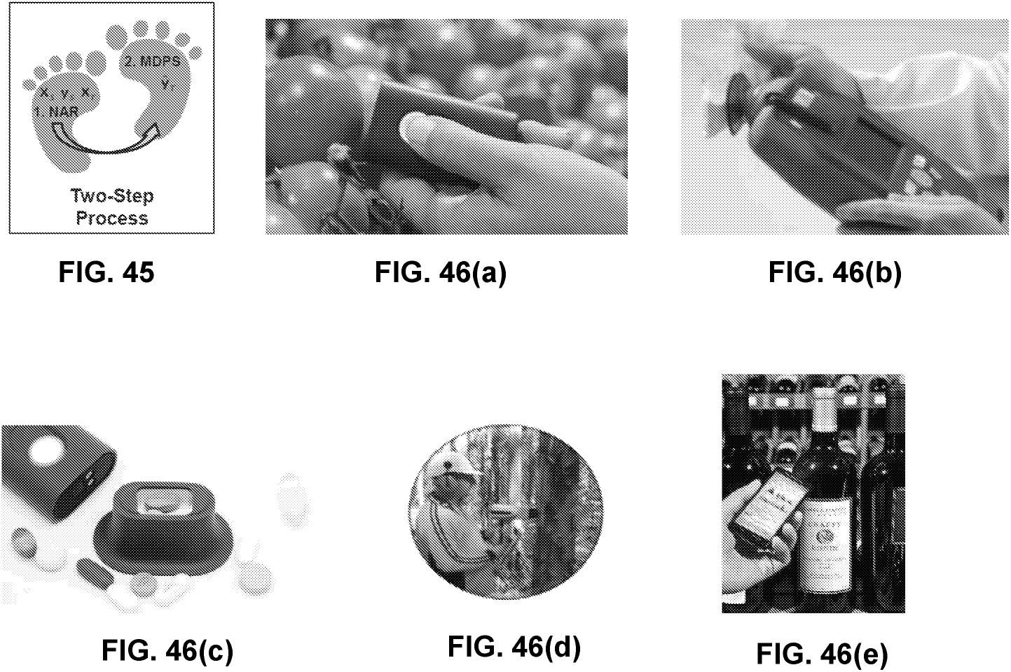

The following relates to exemplary soy seed MDPS model selection histograms. MDPS selects models correlated to lower RMSEV and greater R2 values for all methods up to 3 tuning parameters. MDPS can be used to select primary source models for PLS and RR. The following summarizes Components of NAR with MDPS (as represented by FIG. 44, and the two-step process represented in FIG. 45):
1. NAR provides equivalent RMSEV and R2 values to LMC
   Models are updated without target labels
2. MDPS selects models at or below 1st quartile of prediction errors
3. Models are biased to unlabeled target samples
   a) Used in R to form NAR models
   b) MDPS selects models to predict same unlabeled target samples used in R

In an ongoing context, one may mine historical data for a “target like” primary source sample set (Combine LAFR with NAR for LAF-NAR, and RS-LOCAL: Lobsey CR, et al. Eur. J. Soil Sci. 68 (2017) 840-852). Using automatic analysis with presently disclosed LAFR and NAR, field analysis is more possible in a number of areas, for example, such as cosmetics, clothes, flora, soil, jewels, oils, plastics, pharmaceutical, medical diagnostics, and others, as variously represented respectively by FIGS. 46(a)-46(e). Algorithms may be utilized to relatively quickly show results such as on a user’s mobile phone.

Per the present disclosure, local modeling may be achieved by classification of matrix effects. The following relates to inaccuracy in multivariate calibration. Multivariate calibration succeeds when the target and source library have equivalent sample and measurement conditions, with calibration represented by:

$${\text{y}}_{s} = {\text{X}}_{s} \text{b} \qquad \text{(Eq. 22)}$$

and prediction represented by

$${\hat{\text{y}}}_{T} = {\text{x}}_{T}^{T} \hat{\text{b}} \qquad \text{(Eq. 23)}$$

It is generally understood that matrix effect differences between source and target produce inaccuracy. The following notations are applicable as listed in Table 5:

TABLE 5

Notation

| | |
|---|---|
| X_{S}: | Source library spectra |
| X_{T}: | Target spectra |
| b: | Calibration model |
| y: | Actual analyte |
| ŷ : | Predicted analyte |

Examples of changes in sample or measurement conditions can include:
Different instrument (manufacturer, year, etc.)
Baseline shift over time/Instrument degradation
Novel sample interferents
New human subjects
Sample preparation difference (e.g. pharmaceuticals)

Various sources of inaccuracy may exist such as the target sample is not spanned by the matrix effects of the calibration samples, or the prediction is inaccurate. FIG. 47 graphically represents the cumulative impacts and/or respective contributions of calibration and prediction on resulting validation.

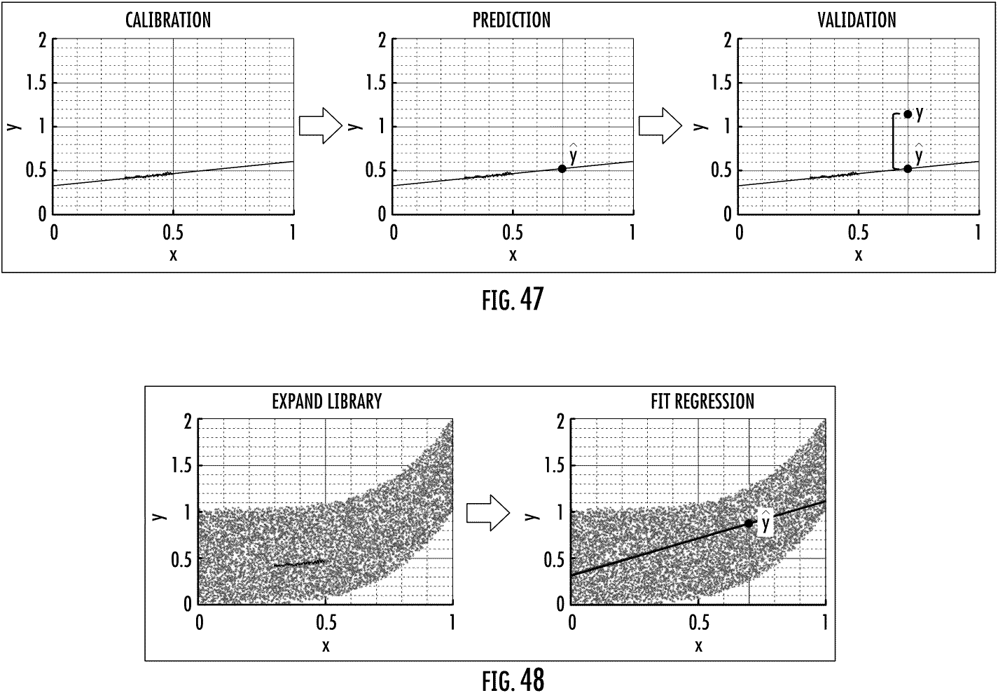

The following relates to potential library expansion. It is viable to expand the library. For example, batch processing, pharmaceuticals, soil scientists all have libraries spanning vast matrix effects. Regarding linear regression fails, this particular model is compromised by too many matrix effects. Prediction is therefore untrustworthy. FIG. 48 graphically illustrates relationship between fit regression and expanded library approaches.

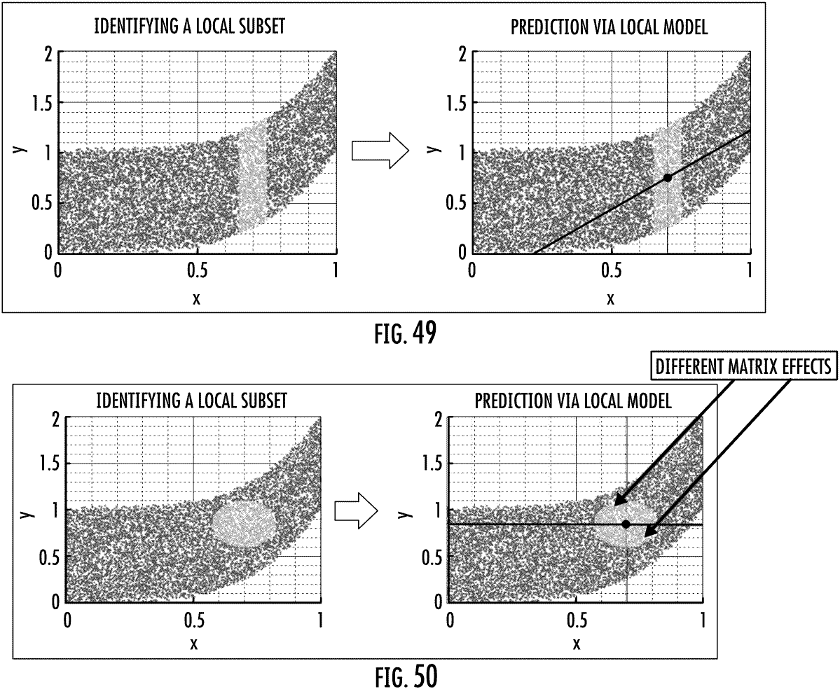

The following relates to generic local modeling. Per the local modeling process, one would find samples which are highly spectrally (x) similar to the target sample, and predict the target sample using only those similar samples. Method examples may include (1) CARNAC: Davies A et al. Mikrochim. Acta 96 (1988) 61-64; and (2) LOCAL: Shenk JS, Berzaghi P, Westerhaus MO, J.Near Infrared Spectosc. 5 (1997) 223-232. Drawbacks may include that samples are only similar in spectra, not necessarily analyte nor matrix effects. FIG. 49 graphically illustrates identifying a local subset in relation to prediction via local model.

The local modeling process involves (1) using the global model prediction to grossly characterize the target analyte, (2) finding local samples which are spectrally (x) and analyte (y) similar to the target, and (3) predicting the target sample using only those similar samples. One method example may include LWR: Naes T, Isaksson T, Kowalski B, Anal. Chem. 62 (1990) 664-673. Drawbacks may include that it (1) uses an often poor global model to characterize analyte similarity and (2) final calibration samples are not similar to one another, only similar to target sample. Additional drawbacks may be that (3) it requires user-dependent input parameters to obtain best results, and (4) the best similarity criterion for multivariate data is not known. FIG. 50 graphically illustrates identifying a local subset in relation to prediction via local model resulting in different matrix effects.

The disclosure herewith includes a new paradigm for local modeling. The ideal local model should have (1) dense analyte distribution tightly spanning target true analyte (similar y) and (2) matrix effects consistent and equivalent to target matrix effects (matrix effects similar). To consider a classification approach: (1) suppose all matrix effects each sample in a dataset are known, (2) construct classes out of different matrix effects, (3) classify the target sample into the best matched matrix effect, and (4) predict the target sample using this class of consistent matrix effects. However, the matrix effects are not labeled. The presently disclosed Local Adaptive Fusion Regression (LAFR) process solves this by:
1. Clustering the library into pseudo-labels of matrix effects
2. Classifying target sample into best matched cluster
3. Predicting target sample using this cluster

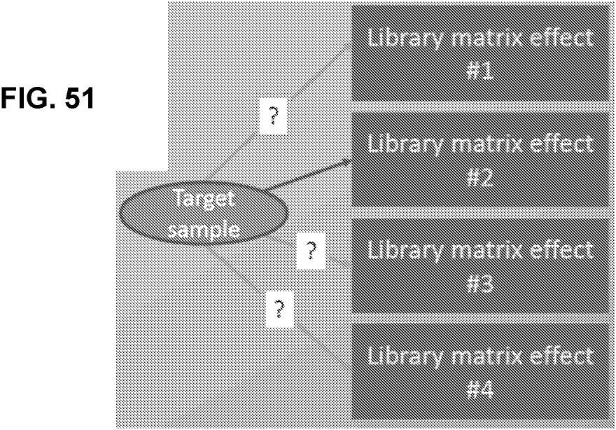

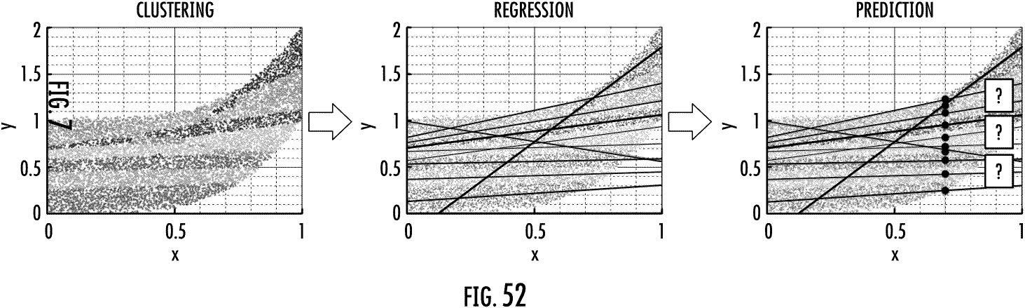

FIG. 51 diagrammatically illustrates an aspect of the presently disclosed Local Adaptive Fusion Regression (LAFR) process. FIG. 52 graphically illustrates presently disclosed relation between clustering, regression, and prediction facets.

When considering how to cluster by matrix effects, one may use Beer’s Law: a calibration set with consistent matrix effects is linear between X and y. Thus, one may use linear clustering to group samples by matrix effects. There may be some initial issues with this approach. For example, there are as many predictions as there are clusters, so one must decide which prediction to trust. Furthermore, one needs a similarity criterion to assess which matrix effect cluster the target sample is matched to.

The following relates to presently disclosed assessment of sample similarity. There generally is necessity for a similarity criterion. For example, LAFR needs to assess similarity for clustering and classification, and real spectral data is multivariate making similarity assessment complex.

The following relates to the presently disclosed Indicator of System Uniqueness (ISU). As disclosed herein, ISU:
Compares a sample to a calibration set (cluster)
Leverages the model regression vector to assess matrix effect similarity
Holistically characterizes similarity using many merits
   17 analyte (y) similarity measures [leverages regression vector]
   15 spectral (X) similarity measures

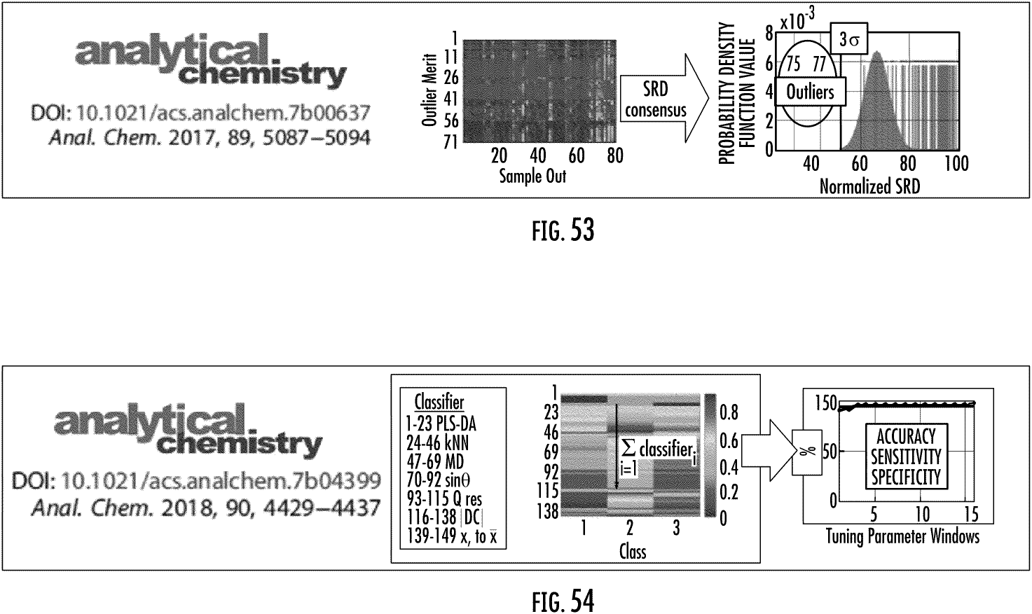

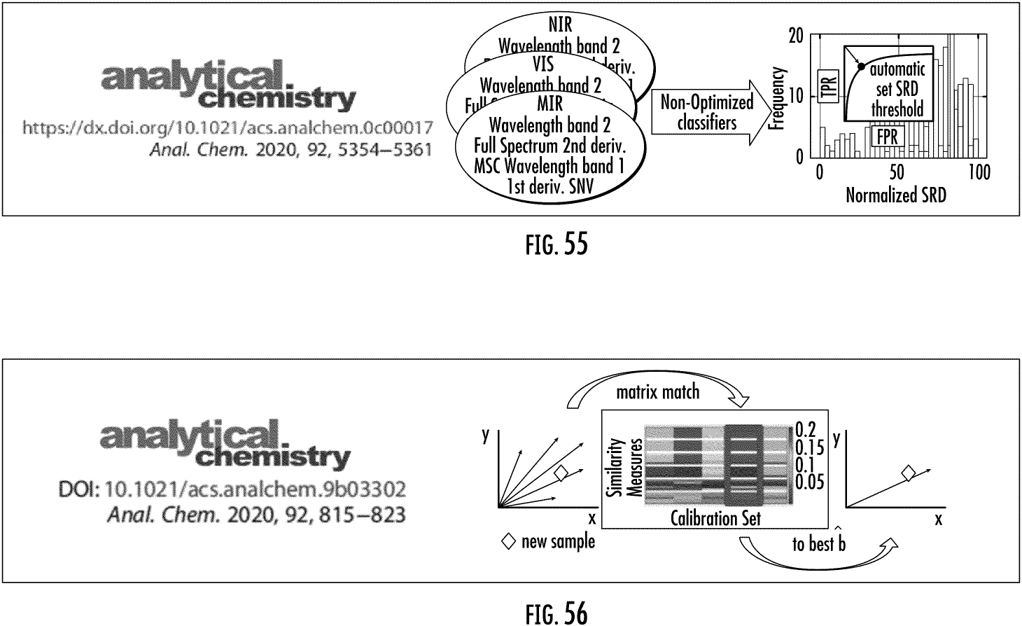

Advantages of the ISU Criterion may be regarded as follows. In the context of spectral similarity measures, outlier detection may be achieved without tuning parameter selection, as graphically represented by FIG. 53. Further, consensus classification may be achieved using non-optimized classifiers, as graphically represented by FIG. 54. Additionally, self-optimized one-class classification (class modeling) may be achieved, as graphically represented by FIG. 55. In the context of “analyte” similarity measures, model regression vectors may be leveraged for matrix matching, as graphically represented by FIG. 56.

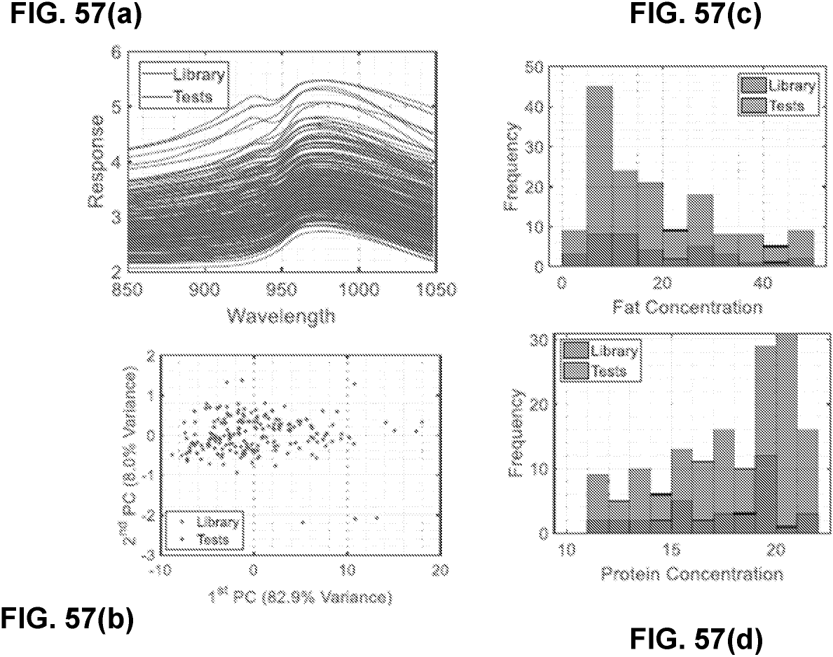

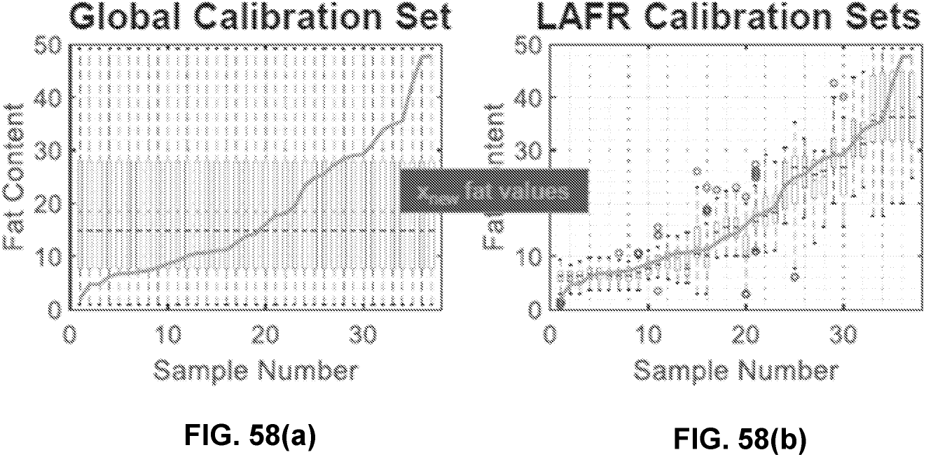

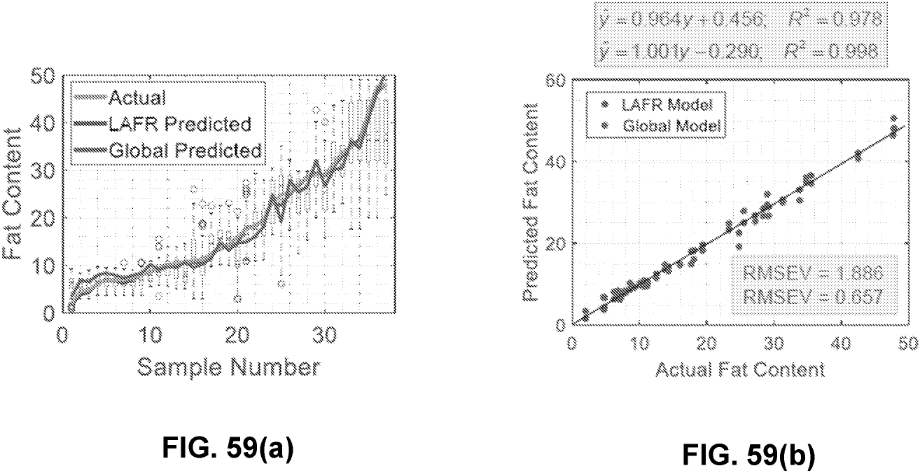

Figure Library info from: Borggard C, Thodberg HH. Anal. Chem. 64 (1992) 545-551, FIGS. 57(a) - 57(d) graphically illustrate a meat data example. In particular, FIG. 57(a) illustrates spectra from 850 - 1050 nm (100 wavelengths), while FIGS. 57(b) through 57(d), respectively illustrate moisture, fat, and protein. There are 156 library samples (global calibration) and 37 target samples. The overall goal of the example is to identify a matrix matched calibration set (model) for each target sample. Regarding the fat content of the target samples, there were 10 minutes per sample, 12 parameter sets, and the LAFR calibration sets are y_{fat} matched to y_{fat} in each x_{new}. FIGS. 58(a) and 58(b) graphically represent the global calibration set and the LAFR calibration sets as to fat values. FIGS. 59(a) and 59(b) involve the LAFR calibration sets locally analyte matched to target, and graphically illustrate that the results of the LAFR predictions are more accurate than global predictions.

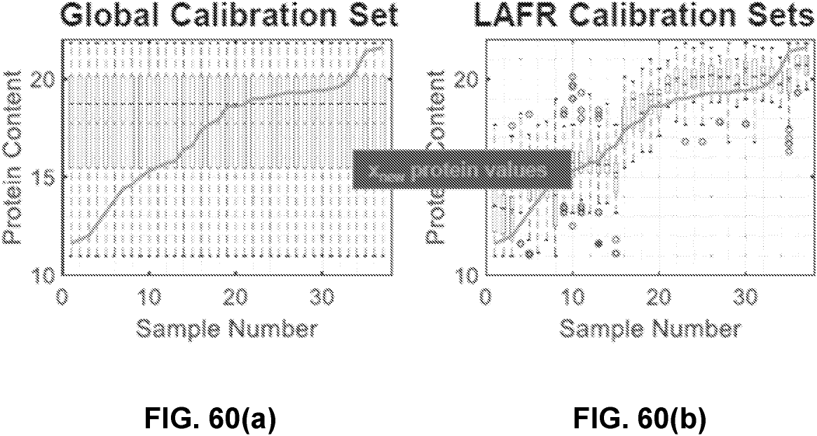

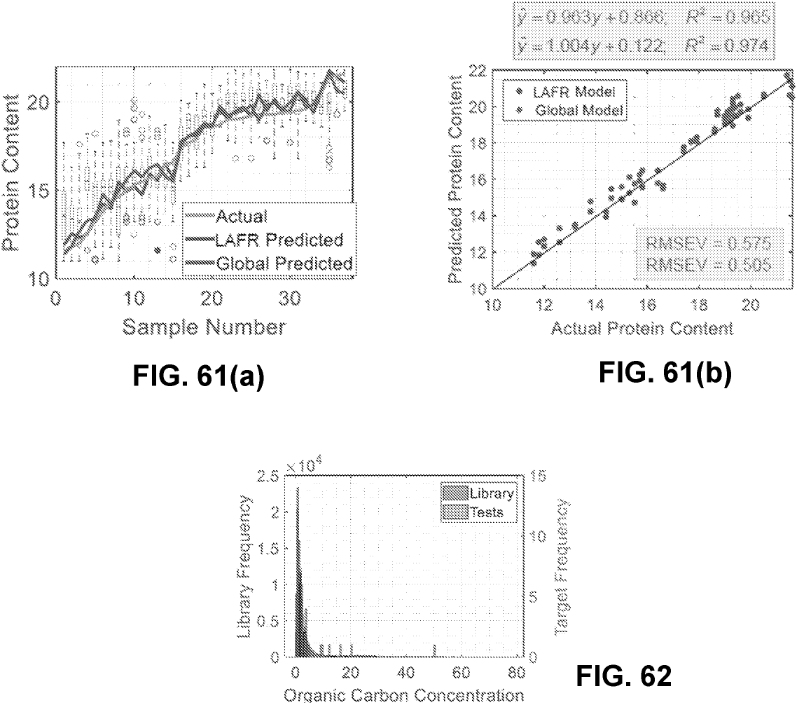

Regarding the protein content of the target samples, there were 10 minutes per sample, 12 parameter sets, and the LAFR calibration sets are yₚᵣₒₜₑᵢₙ matched to yₚᵣₒₜₑᵢₙ in each x_{new}. FIGS. 60(a) and 60(b) graphically represent the global calibration set and the LAFR calibration sets as to protein values. FIGS. 61(a) and 61(b) involve the LAFR calibration sets locally analyte matched to target.

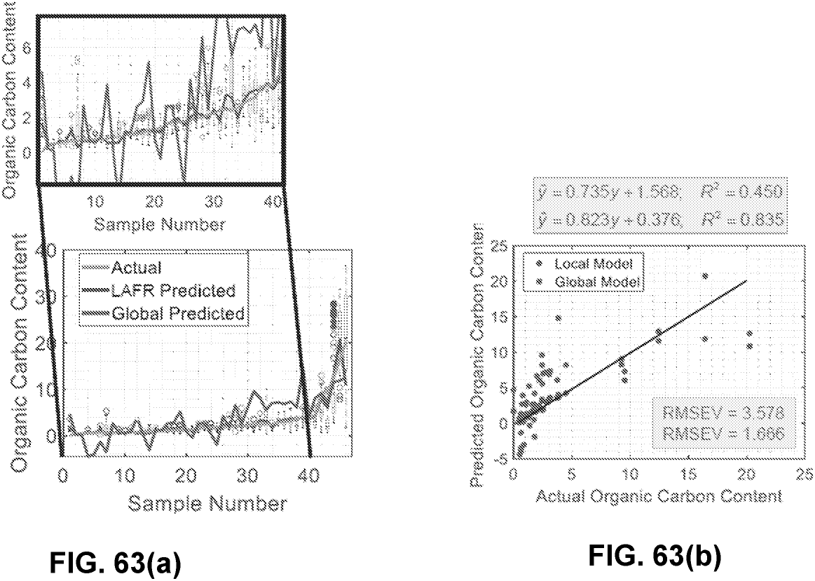

The following relates to a soil data based example, figure from the Rapid Carbon Assessment Project (RaCA), USDA. Spectra was from 350 - 2500 nm (and involved 308 wavelengths), relating to SOC (soil organic carbon), using 98,836 library samples (global calibration) and 50 random target samples. Figure library source from Wijewardane Yufeng Ge, NK, Wills S, Loecke T. Soil Sci. Soc. Am. J. 80 (2016) 973-982, FIG. 62 graphically illustrates comparisons of such library data and tests (targets). Consideration of the target samples for SOC Content made use of Intel (R) Xenon (R) Platinum 8260 CPU at 2.40 GHz (48 cores) and 256 GB RAM. There were 2 hours per sample, 6 parameter sets, and LAFR calibration sets were ysoc matched to ysoc in each x_{target}. Calibration sets are highly localized to target analyte, and LAFR predictions are better than global (LAFR predictions strong and much better than global), as graphically represented by FIGS. 63(a) and 63(b). As seen, four target samples are not predicted by LAFR.

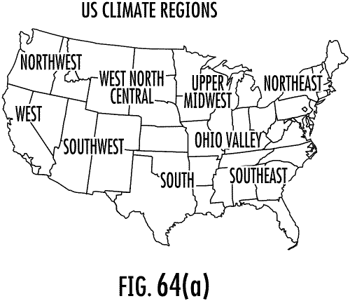

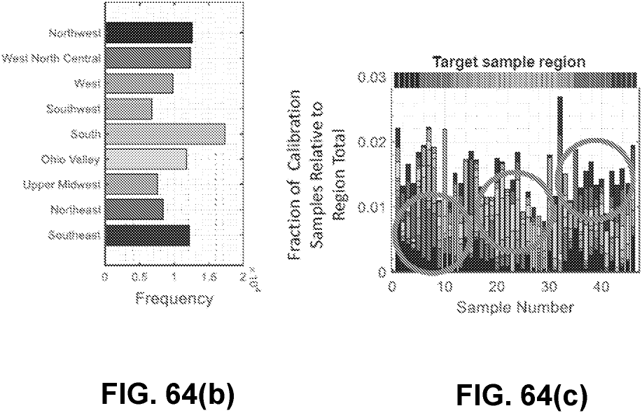

US Calibrations samples tend to be from US regions, as respectively represented by FIGS. 64(a) - 64(c), illustrating US climate regions, frequency of calibration samples by region, and fraction of calibration samples relative to region totals.

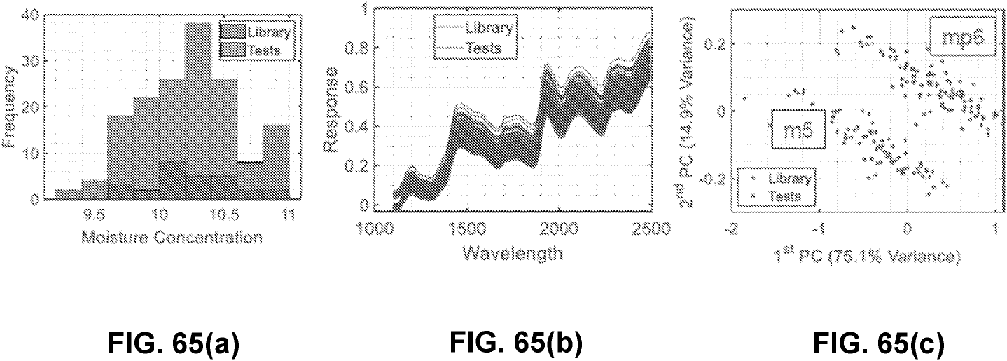

The following relates to a corn data based example, figure as a library source from: Wise B M, Gallagher NB. Eigenvector Research, Manson, WA. http://www.eigenvector.com/data/index.htm The corn data involved target mp5 instrument out of library, with spectra from 1100 - 2500 nm (700 wavelengths). Data related to moisture, oil, protein, and starch. Data involved 160 library samples (global calibration), involving instruments m5 and mp6. The 30 random target samples involved instrument mp5. The presently-disclosed LAFR process should: (1) form mp6 and m5 clusters (calibrations sets) and (2) select mp6 calibration sets and samples as best matched. FIGS. 65(a) - 65(c) respectively graphically illustrate moisture concentration, wavelength, and principal component (PC) comparisons for such corn-related data.

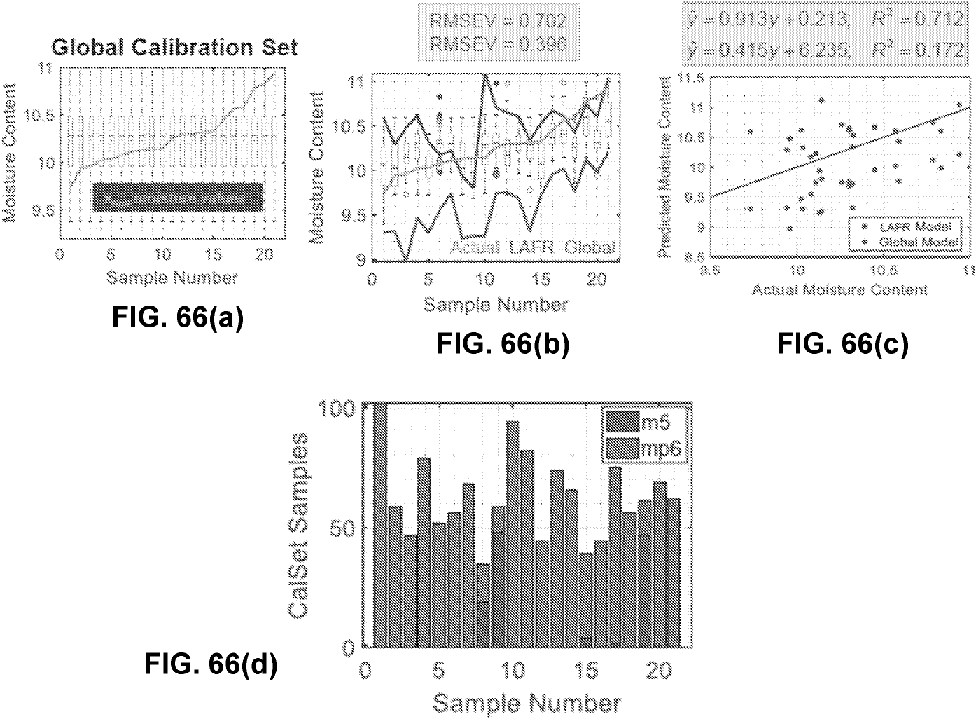

FIGS. 66(a) - 66(d) graphically illustrate various analytic results, using 30 minutes per sample, with 6 parameter sets, variously comparing as indicated global model data, actual data, and LAFR data. LAFR identifies mp6 as best matched instrument to mp5. The Global predictions have a bias due to including m5. There were 9 target samples which were not predicted by LAFR, as shown. Part of the impact to the exercise may be that the associated library is sample limited.

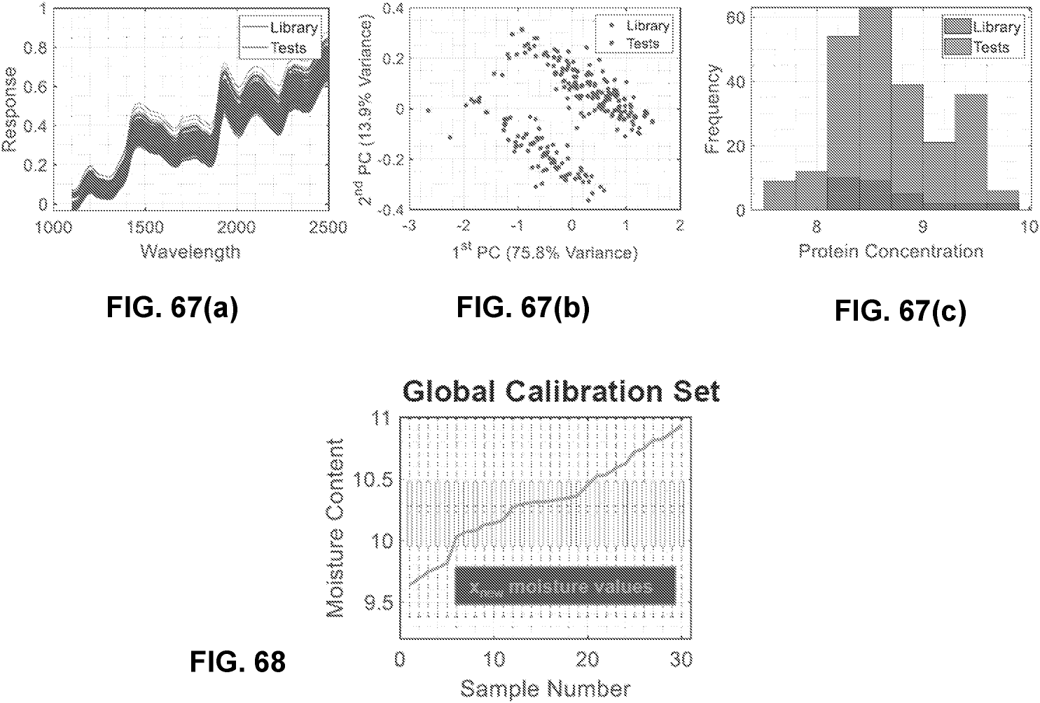

Another set of corn data was considered, with all Instruments in Library. Data spectra was from 1100 - 2500 nm (700 wavelengths), relating to moisture, oil, protein, and starch. The 240 library samples used for global calibration involved instruments m5, mp5, and mp6. The 30 random target samples drew 10 from each of such three instrument. The presently-disclosed LAFR process should: (1) form mp6/mp5 and m5 clusters (calibrations sets) and (2) select calibration samples from respective instrument of origin. FIGS. 67(a) - 67(c) graphically illustrate associated wavelength, principal component (PC) comparisons, and protein concentrations data.

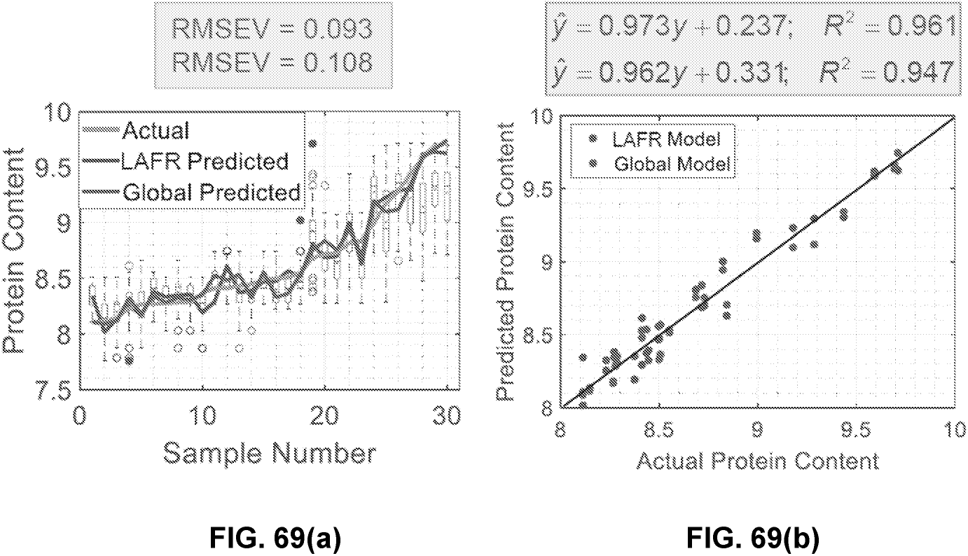

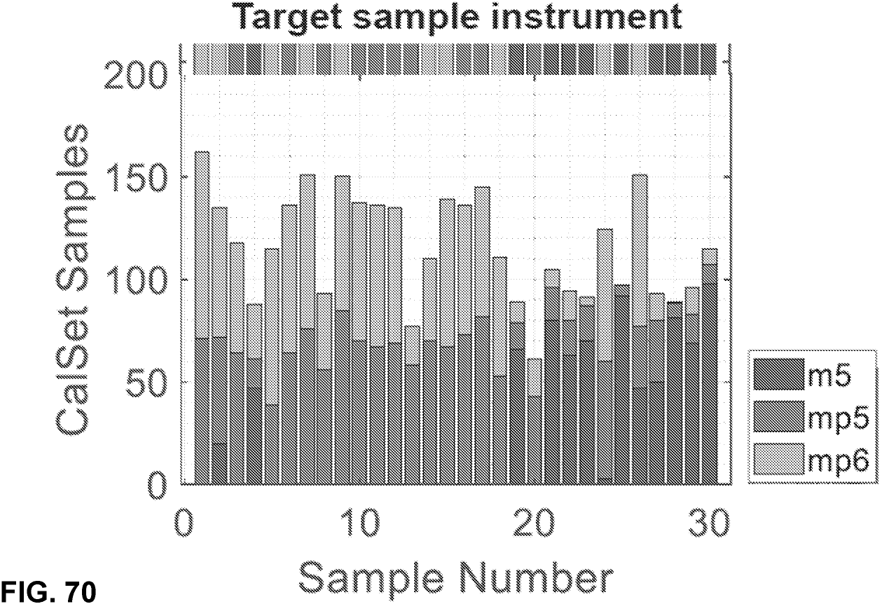

FIGS. 68, 69(a), 69(b), and 70 variously graphically illustrate data respectively associated with moisture values for global calibration set (FIG. 68), protein content for data re Global predicted, actual, and LAFR predicted (FIG. 69(a)), actual versus predicted protein content data (FIG. 69(b)), and m5 versus mp5 versus mp6 comparison data per the target sample instrument (FIG. 70). Such data involved 1 hour per sample, with 12 parameter sets. As illustrated LAFR prediction and global prediction performed equivalently. LAFR effectively recognized the instrument of sample origin, and all target samples were predicted by LAFR. As typical, there may be a consideration of whether the associated library is sample limited.

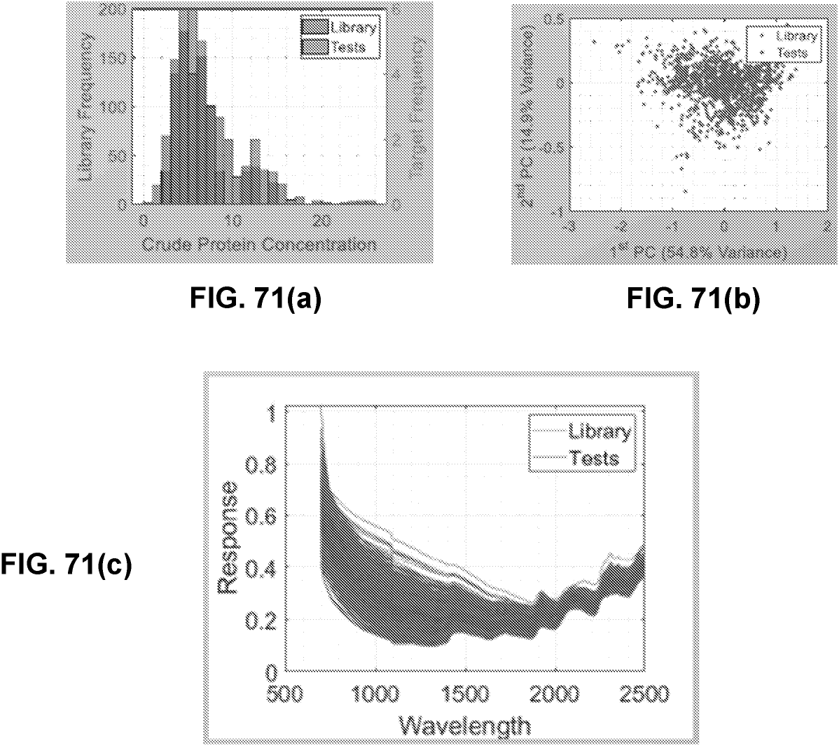

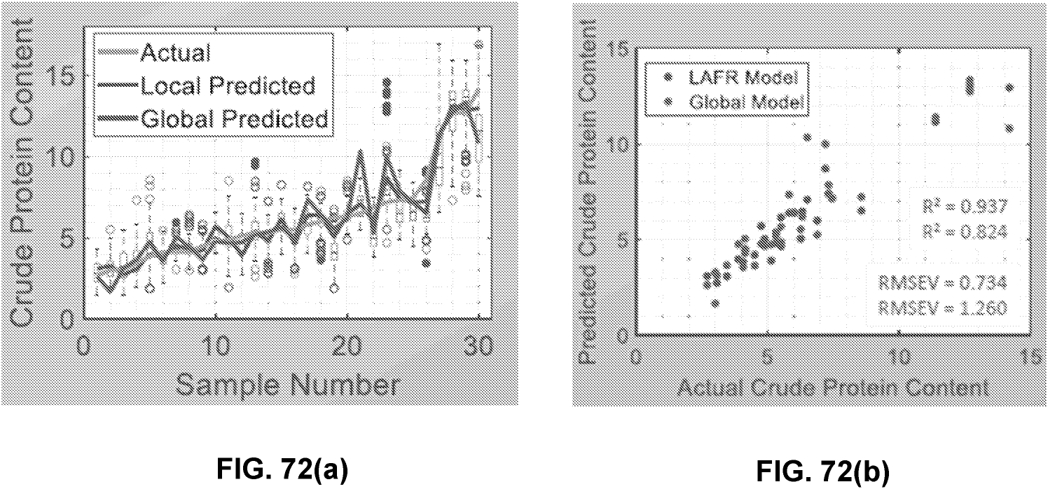

Another presently disclosed example is based on consideration of cattle feces data. Library data source was drawn from Coates DB, Dixon RM. J. Near Infrared Spectrosc. 19 (2011) 507-519. Data involved North Australia 10 year collection with 3 sampling methods: (1) Penned cattle fed freshly harvested pasture, (2) Penned cattle fed forage hays, and (3) Grazed pasture. Spectra data was from 700 - 2492 nm (225 wavelengths), and focus of this exercise was on crude protein. A total of 1172 library samples were used for global calibration. There were 30 random target samples. FIG. 71(a) graphically illustrates crude protein concentration in library versus target compared frequencies. FIG. 71(b) graphically illustrates library versus test data for principal component (PC) comparisons. FIG. 71(c) graphically illustrates the response and wavelength comparisons for library versus test data. FIG. 72(a) graphically illustrates crude protein content for data re Global predicted, actual, and local predicted, while FIG. 72(b) graphically illustrates actual versus predicted crude protein content data. As shown in conjunction with the cattle feces example, focusing on crude protein content, the calibration sets are dense and matched by true analyte, and the presently disclosed LAFR process predictions are better than those of the Global prediction.

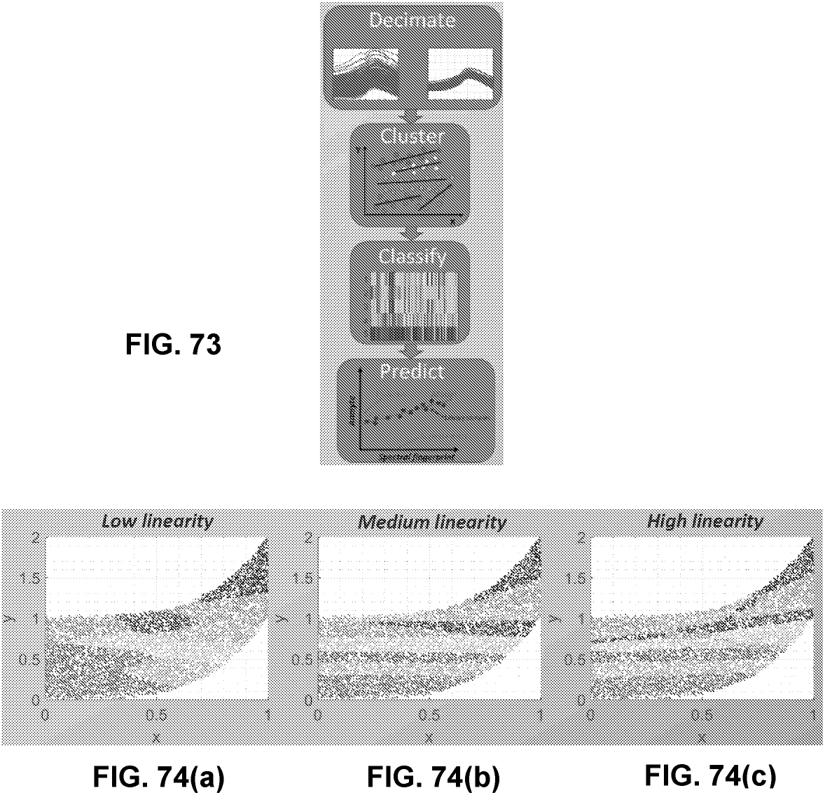

As discussed herein, the presently-disclosed Local Adaptive Fusion Regression (LAFR) Process involves steps of Decimate, Cluster, Classify, and Predict, and as represented by the flowchart of FIG. 73. The decimate step involves decimating the library data into manageable size. The cluster step involves partitioning the library into linear clusters. The classify step involves matrix matching target sample into the most similar set. The predict step involves predicting the target sample using the selected (most similar) set.

In accordance with presently-disclosed subject matter, there can be many clustering options. For example, there are multiple parameters to form matrix-effect-cognizant clusters. It is also to be understood that the best parameter combination may be rarely known to the user. For example, FIGS. 74(a) through 74(c) graphically illustrates the effects of low linearity versus medium linearity versus high linearity. Thus, one aspect relates to how to choose which clustering mechanism gives best prediction.

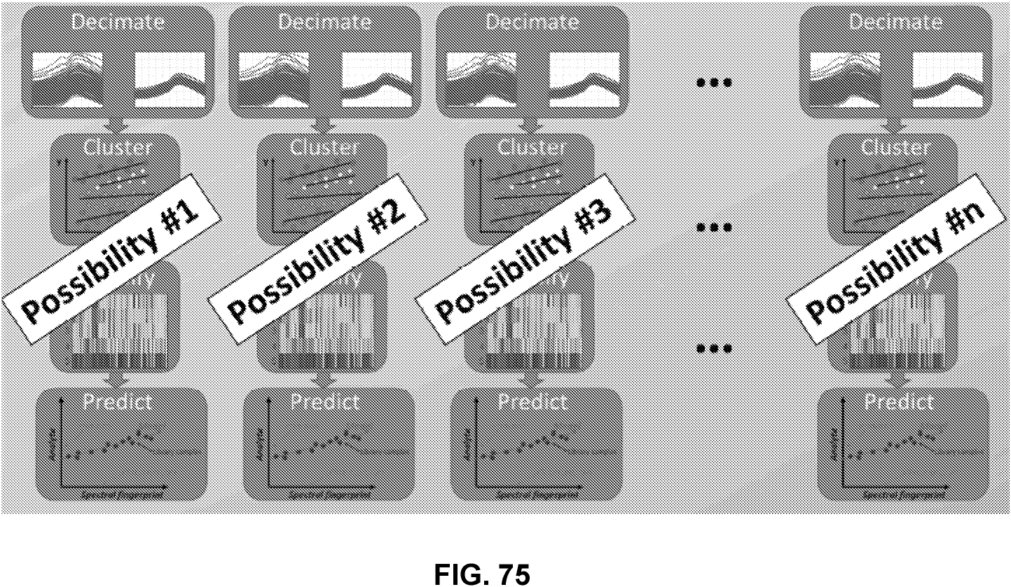

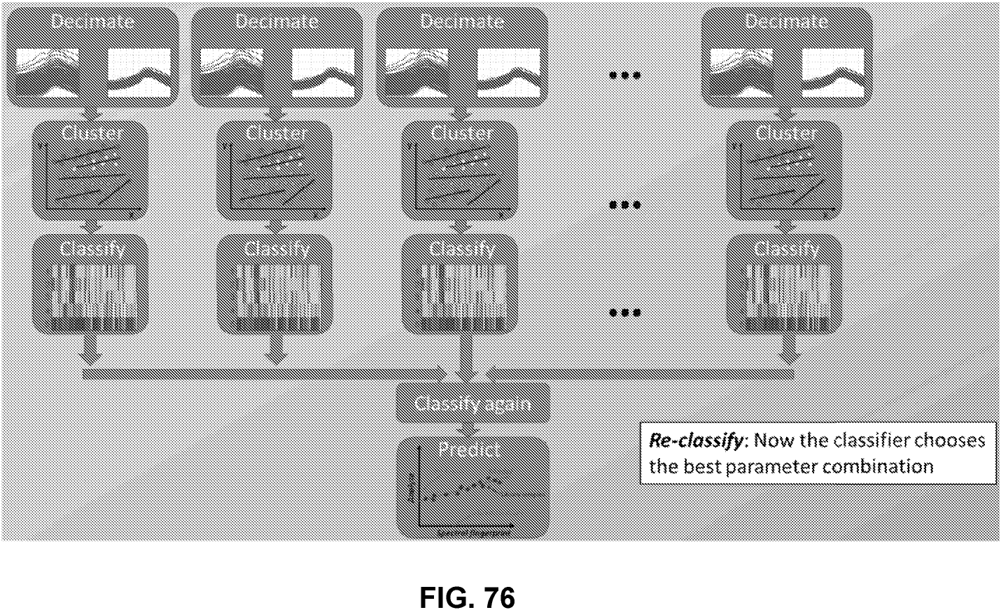

FIG. 75 represents how the Local Adaptive Fusion Regression (LAFR) Process steps Decimate/Cluster/Classify/Predict can be considered over n different possibilities. Then, FIG. 76 represents per presently-disclosed subject matter how to approach re-classifying (classify again substep in FIG. 76) where the classifier chooses the best parameter combination.

As shown herein, the presently-disclosed Local Adaptive Fusion Regression (LAFR) paradigm or process solves issues faced by other local methods. For example, selected calibration set analyte distributions are dense. Analyte similarity is characterized without using a global model. The presently-disclosed indicator of system uniqueness (ISU) provides a holistic characterization of sample similarity. LAFR self-optimizes to find the best clustering parameters. Further, LAFR is shown to be robust to many unique datasets. Additionally, the presently-disclosed clustering-classification approach produces clusters of actual matrix effects. Per future variations, for example, the process of Cluster-Classify-Regress may become Cluster-Classify-Update to handle more complex data situations, such as combination of LAFR with NAR for LAF-NAR.

The following outlines an overview and/or summary of one particular embodiment of the presently disclosed Local Adaptive Fusion Regression (LAFR) process or algorithm, which is extraordinarily complicated in comparison to other contemporary chemometrics modeling methods. This section of disclosure attempts to completely describe the implementation of the LAFR process to a degree that results could be replicated without referring to the original source code. We lay out this section in the following manner.

One important piece of terminology is the concept of a hyperparameter. Hyperparameters control the flow of the LAFR algorithm; they specify how each of the protocol should be carried out, from the similarity merits to use, to the data fusion method, to the number of samples or sets each process should complete with. The LAFR process or algorithm can have a number of hyperparameters (for example, 31) that the user has control over. Despite being possible to alter, however, most of these parameters are kept fixed throughout all the analysis and are generally not expected to be changed by anyone except the most ambitious user.

We define the hyperparameter combination as the unique set of all 31 hyperparameter values which describe one particular runtime protocol for the LAFR algorithm. For example, if one wishes to alter the number of samples per cluster from 20 to 30, there would be two hyperparameter combinations, one associated with the hyperparameter configuration with 20 samples per cluster, and one which is unchanged except for having 30 samples per cluster. LAFR is founded on the principle of iterating over many (generally 10-20) hyperparameter combinations to locate the most optimal configuration for each target sample. The LAFR process or algorithm is set up to accept the variable hyperparameters a user would like to include in their search space, then sets all the other hyperparameters to their default values. A detail which is important to the computational speed but not to the results is that the hyperparameter combinations are organized such that two hyperparameter combinations which differ only in the runtime of the later stages of the algorithm will be grouped together, so that the early stages of the algorithm do not have to be recomputed. Recomputing all these statistics will simply increase the computational time and has no affect on the algorithm outcome.

This section of disclosure also judiciously refers to sample similarity analysis and similarity merits. The foundation for this work is in the Physicochemically Responsive Integrated Similarity Measure (PRISM). It groups the assessment of sample similarity into two categories: spectral similarity and model-informed or analyte-related similarity. All similarity merits in this work are grouped into one of those two categories. In the most general sense, these similarity merits measure some form of similarity between a sample and a sample subspace (note, though, that in some cases only a single sample describes this subspace). If one uses this similarity merit to compare one sample to a subspace, and then another sample to that same subspace, they can characterize the difference in these measured values, which can be interpreted as the difference in how two samples respond to a particular similarity analysis. In the PRISM sense, we term this type of similarity characterization as Δ-similarity. Both standard similarity and Δ-similarity are used many times in the LAFR algorithm. Similarity merits can be further sub-grouped as to whether they require a subspace decomposition to solve. This includes such merits as Mahalanobis distance and Q-residual, that both use principal component decomposition to measure. These merits are naturally more computationally expensive than their simple vector-to-vector counterparts.

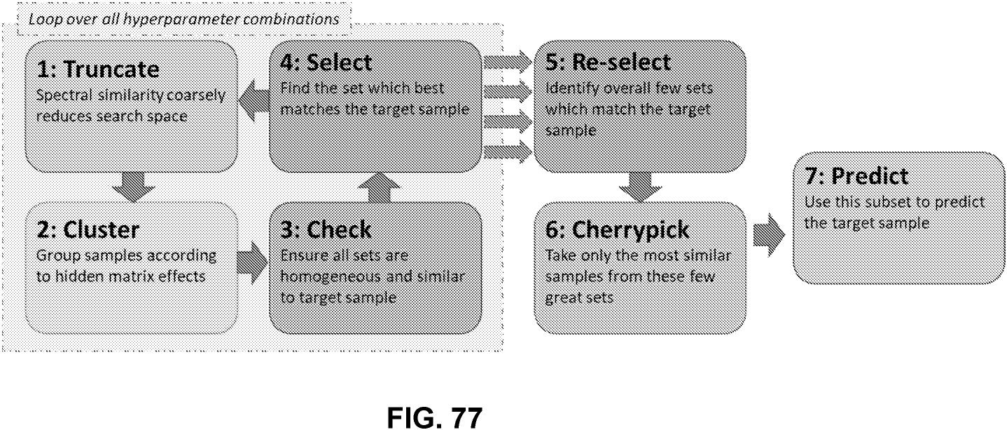

Implementation Details: The LAFR process or algorithm can in some embodiments be coarsely grouped into seven stages. These are: truncation, set formation (or clustering), quality checking, set selection 1, set selection 2, sample selection (or cherry-picking), and prediction. The first four of these occur for each hyperparameter combination; that is, for every hyperparameter combination there are many calibration sets formed, then the best matrix matched set is selected. Once a best set is identified from each hyperparameter combination, the set selection 2 chooses a subset of those to be passed forward into the sample selection, where only the best samples from the input sets are then assembled into a calibration set. This calibration set then predicts the target sample. This entire program flow is depicted in FIG. 77.

Though the overarching idea for the LAFR process or algorithm is fairly straightforward, the details of its implementation require quite a bit more explanation. For example, there is an intermediary outlier detection step between the truncation and clustering, the clustering requires quite a bit more theory, and the quality checking process has many sub-steps. The following sections are dedicated to describing these intricacies in detail; however, this flowchart in FIG. 77 should always be referred to as the overarching program flow.

Truncation: The idea behind truncation is to reduce the size of the library by a sufficient amount so that clustering is computationally feasible. It aims to eliminate the samples from the library which have negligibly minimal likelihood to be matched to the target sample, and so they are simply cumbersome to the analysis. This is, however, only a concern in medium and large datasets. In spectral libraries with less than 500 samples, it is likely that clustering is computationally viable on the whole library and could be performed as such. Library reduction can be performed in one or both of two ways: categorical truncation and similarity-based truncation.

Categorical truncation uses known information about the target sample to restrict the local modeling search space only to library samples which are matched in that known information. Take for example the case of known geographical region of origin of a set of samples. A new target sample likely should be predicted best by the samples within its same geographical region; this is a category which one could truncate by. However, categorical truncation can only occur if the same categories are measured both on the target sample and on the library samples, and if this known categorical information is strongly correlated with true sample similarity-not restricting the analysis only to samples which are not necessarily any more similar.

Categorical truncation in LAFR is simple: identify library samples which are matched in all the same categories as your target sample. It is possible that there are multiple different categories one could measure; the truncated set should be matched by all of these. Hyperparameters associated with categorical truncation are “TruncCategorical” which determines whether categorical clustering should take place, and “TruncCategories” which specifies which categories to truncate according to. Once this categorical truncation is carried out, then the user can either move forward into the next stage or truncate even further by a similarity-based analysis.

Whether categorical truncation was carried out or not, the next step which can occur is similarity-based truncation. The basic idea is to use computationally inexpensive similarity merits to identify which library samples are similar to the target sample. Of the similarity merits used in this work, the least computationally expensive ones are the spectral similarity merits which do not use principal components.

The exact implementation of this similarity-based truncation in this exemplary embodiment is as follows. Calculate the vector-to-vector similarity directly between the target spectrum and each of the spectra in the sample library. Normalize the similarity merit measurements such that the vector containing the similarity values for all the library samples for a particular merit has unit magnitude (i.e., divide each value in the vector by the magnitude of the whole vector). Sum over all the similarity merits for each sample so that every sample now has one score containing aggregate information from all the similarity measurements. Finally, choose the samples with the lowest fusion score (i.e., the samples which are most similar to the target sample) to continue on to the next stage. The number of samples selected is a hyperparameter which is also described in more length in the appendix.

Library Outlier Cleaning and Detection: Another one of the guiding principles in LAFR in this exemplary embodiment is that, at certain times in the algorithm after particularly tumultuous operations, the data should be swept for outliers. The idea is that having outliers constantly within the data can lead to propagated error in the similarity analyses in particular. By analyzing the data for outliers, cleanliness of data throughout the algorithm can be assured. The outlier analysis in this section is two-fold: first, clean the library (i.e., remove any outliers from the truncated library), then detect whether the target sample is an outlier to this new truncated library. It is quite possible (and frequently observed) that the target sample conditions are not spanned by the conditions of the library samples and is thus an outlier to the entire library. This section of the disclosure aims to detect this situation.

Cleaning the library and detecting whether the target sample is an outlier is fundamentally the same problem as each other. One must simply determine whether a sample of interest, whether it be a library sample or the target sample, appears to be an outlier with respect to the rest of the library samples. Firstly, the sample of interest is separated from the rest of the library. All the spectral similarity merits, including the ones which require a principal component decomposition, are used to assess the similarity between the sample of interest and the remaining library samples as a whole. For the vector-to-vector similarity merits, this means calculating the similarity between the spectrum of interest and the centroid (mean spectrum) of the remaining library samples. For the subspace similarity merits, such as Mahalanobis distance, this similarity between a sample set and spectrum of interest is already well defined and is performed here. The resulting similarity data from this type of measurement is three dimensional: number of similarity merits by number of library samples (plus one for the target sample) by the number of principal components. To fuse this data, first average over all the principal components, then normalize each set of measurements at one similarity merit to unit magnitude as was done in the truncation stage. Finally, sum over the normalized similarity measurements for each sample. Resulting, then, is a collection of numbers describing how each library sample and target sample is similar to the remaining library samples when it is removed from the set and compared.

A simple way to use this fusion similarity data in detecting outliers is as a z-score. The measurements of the library samples with respect to the remaining library is used to find a standard deviation and a mean. Samples with a low fusion score are highly similar to the remaining space, and samples with a high fusion score are very dissimilar to the space (outliers). A hyperparameter controls the number of standard deviations from the mean before a sample is considered to be an outlier. This value is by default -3, meaning that outliers have a fusion score that is more than three standard deviations greater than the mean of the library samples in general had. It should be noted that this z-scaling is based on the standard deviation and mean of the library samples and did not include the target sample. This is since skew due to the target sample being an outlier could have an undesirable effect, and library samples are already considered to be reasonably outlier-free compared to the target sample effect.

If the target sample is identified to be an outlier (i.e., more than the specified number of standard deviations from the mean), then the sample is deemed “non-predictable” and LAFR will decline to provide a prediction for this particular target sample. If a user strongly desires to achieve a prediction, likely resulting in much inaccuracy, then they can alter the outlier detection z-threshold to facilitate a prediction.

Since some of the similarity merits use a principal components decomposition of the library spectra, it is necessary to discuss how the number of principal components is selected. Generally, the selection is carried out using a 99% variance rule; the library spectra are mean-centered, and a singular value decomposition (SVD) is taken. The maximum number of principal components is determined by the cumulative sum of the singular values to achieve 99% of the total sum of the singular values. Similarity analysis is calculated using the window of principal components (from only one principal component all the way to the maximum number), then averaged over as described above. This will be the case for all the applications in this algorithm which use principal components analysis.

Set Formation: The set formation (or clustering) in this exemplary embodiment of the LAFR process or algorithm is described as grouping the samples by matrix effects, or homogenizing the calibration sets with respect to their matrix effects. We know that sample clusters which are sufficiently homogeneous in their matrix effects will have a linear (i.e., Beer-Lambert type law) relationship between their spectrum and analyte amount. This can be reverse-engineered to realize that linear calibration sets are more likely to be homogeneous in their matrix effects. The task of creating matrix-effect-grouped calibration sets is then mapped onto the problem of creating linear clusters, which is an active field of research across different implementations of machine learning.

We employ a modified cluster-wise linear regression approach that simultaneously optimizes calibration sets to be spectrally similar, analyte similar, and maintain linearity. Linearity is ensured by using prediction error of the sample. If it is not self-evident why this is the case, then this research is all laid out in other linear clustering literature [referenced in the introduction]. The overarching idea is that clusters are initialized, then iterated, reassigning all samples on every iteration to the cluster which they are most similar to by a weighted combination of spectral similarity, analyte similarity, and ability for that cluster to predict the sample accurately.

The first step is to initialize the clusters. Alterations in cluster initialization processes substantially impact the outcome of the converged clusters. The simplest and most widely employed cluster initialization tactic is to randomly assign samples to all the clusters. This option is possible in our algorithm but is not the default as specified by the controlling hyperparameter. Instead, we opt for a Kennard-Stone approach in which the cluster centroids are the center sample of the library, and then the remaining cluster centers are the outermost samples. These centroids should have maximal intra-cluster variance possible within the dataset. The Kennard-Stone algorithm typically uses only Euclidean distance, however we modify the algorithm to weight with equal importance the Euclidean distance between samples as well as the actual analyte difference. This Kennard-Stone process provides centroids to use, however the next part of the clustering process requires that these be entire clusters, not simply centroids. To accomplish this, the remaining samples are classified onto the cluster they are most similar with according to a spectral and analyte similarity measure. For the spectral analysis, all the vector-to-vector spectral similarity merits (the same as truncation) are used. The analyte similarity analysis is simply the difference in true analyte between the unassigned sample and the centroid. Each unassigned sample is compared to the centroids using the spectral similarity merits and the analyte difference. All the measurements at a particular merit are normalized to unit length, as always. Then the spectral similarity measurements are averaged over for the samples, then this amount is averaged with the analyte similarity measurement to produce an overall similarity score for each unassigned sample to each cluster. Each unassigned sample is then assigned to the cluster which it has the lowest similarity score to.

Now that the initialized clusters exist, either by randomly assigning samples or by a Kennard-Stone plus simple similarity analysis, the k-means based linearization can occur. All the samples are up for reassignment, meaning that they will go from their current cluster potentially to a new cluster. The similarity merits compare the samples up for reassignment to the overall subspaces defined by the prior sample clusters (i.e., the clusters from the last iteration). For the spectral vector-to-vector merits, this means comparing the spectrum up for reassignment to the mean spectrum of the cluster. For the analyte merits, it compares the samples up for reassignment to the mean analyte of the cluster. Many similarity merits are involved in this portion of assessing which cluster the samples are most similar to. The spectral scores for each of the samples compared to each of the clusters, as well as the corresponding analyte similarity scores, are fused the same way as in the other cases (average over principal components or latent variables, normalize merits to unit length, then average over the similarity merits). The last measurement is the linearization condition, which is the prediction error of the cluster model when used on the sample up for reassignment. This is straightforward to calculate, and is also normalized to unit length for all the samples, just as was the case for all the other similarity merits. At this point, there are measurements for spectral similarity, analyte similarity, and prediction error of each of the library samples with respect to each of the clusters from the prior iteration. The spectral similarity, analyte similarity, and prediction error scores are fused together into one composite score using a user defined (hyperparameter) weighting scheme. For weights α₁, α₂, and α₃, this weighted fusion obeys Eq. 24.

$${s}_{c o m p o s i t e} = {α}_{1} {s}_{\text{x}} + {α}_{2} {s}_{\text{y}} + {α}_{3} {s}_{p} \qquad \text{(Eq. 24)}$$

Each sample is then assigned to whichever cluster it has the smallest composite similarity score to. This readjusts the clusters, and then the process can be iterated once again to attempt to converge towards a solution in which all samples are in clusters that they are highly similar to. Convergence, which is defined in this context to mean that no samples change clusters when they are up for reassignment for an iteration, is the premier goal. However, it is typically computationally infeasible to always iterate towards complete convergence since the many similarity calculations required for each step are quite computationally expensive. Instead, a hyperparameter controls the maximum number of iterations (default 30) before those clusters are exported to the next stage. Even without complete convergence, the clusters can still satisfy the linearity constraints to a very high degree.

A debilitating problem with this process is that it almost always encourages the samples to be clustered into calibration sets of uneven numbers of samples. This causes some calibration sets to be so small that they cannot even develop a regression vector and thus cannot be used in the iteration process. Another hyperparameter controls the minimum number of samples required in each cluster for every iteration. If a cluster falls below this minimum number of samples, then the algorithm will split the largest cluster in half (into high analyte and low analyte) and append the high analyte set to the cluster which had insufficient size. This process applies recursively until all the clusters are of sufficient size. Then the next iteration stage can occur as normal.

Set Outlier and Quality Controls: This step exists in this exemplary embodiment to ensure that the formed calibration sets are outlier free, are relatively linear between their spectrum and analyte, and the target sample is fairly similar to them. There are five substages which ensure these conditions are met: Grubb’s test, labeled analyte clean, spectrum and labeled analyte clean, linearity quality control, and unlabeled PRISM check.

The Grubb’s test simply checks whether any of the samples within the calibration set have analyte amounts which are far from the rest of the samples. The confidence interval associated with this Grubb’s test is a controllable hyperparameter, but its value defaults to a 95% confidence interval. If any samples are detected as outliers, they are removed.

The labeled analyte clean uses many similarity merits to assess whether any of the samples in the calibration set are dissimilar enough to be outliers. This analysis uses the PRISM-style of similarity checking which is Δ-similarity. A sample is taken out of the calibration set, call this the sample out. This sample is compared to the remaining calibration subset using all the similarity merits. Also, though, a sample from within the remaining calibration subset is also compared using these similarity merits to the remaining calibration subset-call this the sample left in. Now we have similarity measurements for the sample out to the remaining subspace and the sample left in to the remaining subspace, take the absolute value of the difference of these two measurements and we are left with the Δ-similarity describing the similarity between the sample out and the sample left in. Iterate over all the possible samples left in to get the full data picture. Then, repeat this process for all possible samples left out. What results is four dimensions of similarity data. All samples out against all samples in against all similarity merits against all latent variables. To fuse, first average over the latent variables, then over all the samples left in. Now normalize the similarity merit rows to unit length, and finally sum over the similarity merits. Just as we had earlier for the library outlier detection, there is one similarity value for each sample left out describing its similarity to the rest of the sample space. Just as before, scale this value according to the z-distribution and reject any samples with scores outside the standard deviation threshold.

That outlier detection process only considers the analyte similarity. To more holistically characterize the outliers, then we repeat that exact process with both analyte similarity merits and the spectral similarity merits, with the same fusion process as well as outlier rejection.

The quality controls come next. The idea is that poor calibration sets should be rejected before they even have the chance to go through the similarity analysis in the next stage. The simplest rejection criterion is the calibration set size. If the outlier cleaning has left them with few enough samples (hyperparameter default 10 samples) that a regression is likely inaccurate, then the calibration set is rejected. Next, if the predicted analyte value of the target sample does not fall within both the actual analyte range of the calibration set samples as well as the predicted values of the calibration set samples, then the calibration set is also rejected, since these types of models are generally not optimal for extrapolation. The next few conditions are related to the dataset linearity and are based on fitting a regression line to the univariate plot of the predicted analyte values for the calibration set to the actual analyte values. The R² correlation should optimally be 1.00, the slope of this fit should be 1.00, and the intercept should ideally be 0. The adherence to these optimal conditions is set by a hyperparameter, but generally the only easy one to strictly control which is dataset independent is the R², which we confine to be strictly greater than 0.80 for the default hyperparameter value. Finally, the RMSEC can be constrained by the quality controls, but again this is not dataset independent.

The final part of the checks is to see whether the target sample is an outlier to the calibration set. This is performed the same as the PRISM algorithm, and the same as the aforementioned outlier cleaning mechanism except with some different similarity merits. The merits must be different since analyte amounts are not known for the target sample. Like always, if the target sample is outside of the allowed standard deviation threshold, then it is identified as an outlier.

Set Selection 1: The first set selection of this exemplary embodiment takes the generated calibration sets from this particular hyperparameter combination and identifies the set which is most similar to the target sample. This similarity analysis is relatively straightforward compared to some of the earlier ones. However, it brings up the topic of self-modeling and cross-modeling in set selection, or matrix matching. Self-modeling is the typical use case of similarity merits in the Δ-similarity setup. To calculate the self-modeling similarity, one sample is taken out of the calibration set, and compared to the remaining subspace of the calibration set using the similarity merits. Then the target sample is also compared to this remaining calibration set. The difference between the two measurements for these different samples with respect to the same underlying subspace is recorded as the Δ-similarity, and indicates one measurement of similarity between the target sample and the calibration sample out. This can be performed for all the source samples out, for all the calibration sets. However, recent work introduces the idea of cross-modeling, wherein the sample out and target sample are compared to not the calibration set that the sample out originated from, but rather a different calibration set. For example, if there are two calibration sets, then one can calculate the similarity between a sample pulled from the first calibration set to the entire second calibration set, and also the target sample to the entire second calibration set, then difference them to get a Δ-similarity. This cross-modeling not only expands the volume of accessible data to draw similarity conclusions based on, but also aids in providing a separate view of the internal physicochemical similarity structure between the samples.

So, in order to pick the best matched set, LAFR calculates the Δ-similarity between the sample out and the target sample with respect to not only the set the sample out originated from, but also the domain of each of the other calibration sets. The number of effective similarity merits is expanded multiplicatively by the number of calibration sets, so that there is effectively a “Mahalanobis distance with respect to the first calibration set”, a “Mahalanobis distance with respect to the second calibration set”, etc. To fuse over this data, first it is averaged over principal components and latent variables, then averaged over all the calibration samples left out. Each effective similarity merit is normalized to unit length, then summed over all the similarity merits. At this point, there is one spectral similarity score and one analyte similarity score for each of the calibration sets.

The method to fuse the spectral and analyte similarity scores is also a controllable hyperparameter. The simple “mean” fusion just sums the spectral score and the analyte score. The default value, which is “plot” fusion, instead takes the 2-norm of the coordinate vector defined by each sample’s spectral similarity score and analyte similarity score.

$${s}_{c o m p o s i t e} = \sqrt{{s}_{\text{x}} {}^{2} + {s}_{\text{y}} {}^{2}} \qquad \text{(Eq. 25)}$$

The set with the lowest composite similarity score is selected and passed forward to the second set selection. However, it is at this point that the LAFR algorithm goes back to iterate over the other hyperparameter combinations, yielding a “best” calibration set for each of them. The aggregate of all these calibration sets, one for each hyperparameter combination, will be the input for the second set selection.

Set Selection 2: The second set selection per this exemplary embodiment is almost exactly functionally equivalent to the first one, in terms of implementation. The first difference, though, is that cross-modeling is not used for the second set selection, only self-modeling is analyzed. This is since nearly the same sets can be created by the clustering algorithm, and thus it is frequent that the second set selection has nearly duplicate calibration sets, which severely advantages the sets which happen to be duplicated in the cross modeling. Using only self-modeling abates this issue. The second small difference between the two set selection stages is that the second set selection chooses multiple best calibration sets instead of only one. The idea behind this is that the sample selection which occurs next works most optimally if it selects samples from multiple different calibration sets and aggregates them, rather than selecting all the samples from an already relatively small single calibration set.

Sample Selection: Now that the duplicate calibration sets have ideally been removed by the second set selection, it is appropriate to return to using the cross-modeling statistics. Thus, the self- and cross-modeling measures are computed for the new subset of calibration sets. Recall, though, that earlier the set selection process averaged over the samples left out. For sample selection, however, this fusion will not take place. Instead, when the merits are normalized to unit length and summed over, there will be one spectral similarity value and one analyte similarity value for each sample in the calibration sets, rather than only for each calibration set. As before, the plot fusion in Eq. 25 can be used to combine the spectral similarity score with the analyte-based similarity score.

Another hyperparameter controls the number of unique samples selected by the algorithm to be in the final local calibration set. The parameter specifies that the samples must be unique because equivalent samples often arise in different calibration sets passed to the final sample selection and are chosen. Since some samples will be duplicated in the final calibration set, this works as a de facto weighting scheme whereby samples which are repeated more often will have additional influence on the formation of the model regression vector.

One substantial concern with the last piece of the LAFR process or algorithm is that these final hyperparameters cannot self-optimize because they arise outside of the loop over all hyperparameter combinations. It is certainly possible that minor changes to the latent variable selection method or the number of final chosen samples could substantially affect the predictive capabilities of the LAFR model. To abate this issue, we analyze the variation of the model predictions when the number of unique samples and the number of latent variables is altered. We vary the number of unique samples from 15 less than the hyperparameter value to 15 more and look at the latent variables from 2 less than the chosen amount to 2 more than the chosen amount. This defines a parameter space in which we can analyze all the predictions and determine whether they vary to a degree which is unacceptable for the required predictive reliability. To make this minimally restrictive, the LAFR default hyperparameter for this is that if the standard deviation of the varying predictions is greater than 40% of the total span in the analyte amounts for the whole original sample library, then the sample is identified as non-predictable. This case is almost never triggered, but if the standard deviation is this large then the sample certainly is not reliably predicted by the calibration set.

Prediction: Having determined a local calibration set from the processes prior to this, the prediction of the target sample is straightforward. For the purposes of this exemplary embodiment of the process, partial least squares regression is used, but a variety of linear regression techniques are viable for generating predictions.

While certain embodiments of the disclosed subject matter have been described using specific terms, such description is for illustrative purposes only, and it is to be understood that changes and variations may be made without departing from the spirit or scope of the subject matter.

What is claimed is:

1. Methodology for searching a large library of field sample spectra (Library X, y) using a generalized local adaptive fusion regression (LAFR) process for quantitative analysis of molecular based spectroscopic data (x_{new}) from a target sample of analytes, comprising:
defining LAFR process parameters, including the number of library samples to retain in a decimation step, the number of calibration clusters to form, and the number of fundamental parameters to use;
applying a decimation step to the library to reduce the library to most N spectrally similar to target sample, and to perform an outlier check to remove reduced library components for which the target sample is an outlier;
forming linear calibration sets defined by the LAFR process parameters;
performing an outlier check to remove linear calibration sets for which the target sample is an outlier;
using ISUₓ and ISU_{y} sample-wise similarities to mine the library of field sample spectra with reference amounts for a local training set explicitly matrix-matched to the target prediction sample;
forming a prediction model formed with the local training set; and
using the prediction model to predict the quantitative analysis of a target sample,
where ISUₓ and ISU_{y} sample-wise similarities comprise indicators of system uniqueness (ISU) to assess the degree of matrix matching between reference samples and the target sample.

2. Methodology according to claim 1, wherein ISU holistically characterizes similarity by a fusion of multiple similarity merits.

3. Methodology according to claim 2, wherein LAFR process parameters for the decimation step include spectral similarity measures, and number of samples post-decimation.

4. Methodology according to claim 1, wherein forming linear calibration sets is based on a Beer’s law like linear relationship where a calibration model is made that linearly relates the analyte amount to the measured spectral responses.

5. Methodology according to claim 4, wherein the calibration model is used as the prediction model to predict the analyte amounts present in each target sample.

6. Methodology according to claim 5, wherein the calibration model uses inherent matrix effects as information for training calibration sample sets to successfully predict new target samples.

7. Methodology for searching a large library of spectral measurement (Library X, y) using a generalized local adaptive fusion regression (LAFR) process for quantitative analysis of molecular based spectroscopic data (x_{new}) from a target sample of analytes, comprising:
define process parameters and obtain all possible hyperparameter combinations (HPPC’s) thereof;
for each HPPC:
reduce Library to N most spectrally similar to the target sample,
form calibration sets (CalSets) by clustering analyte ranged windows,
remove all CalSets for which the target is an outlier to produce Approved CalSets,
use matrix matching to identify a Selected CalSet from the Approved CalSets which best matches to the target sample, and
store the Selected CalSet for each HPPC;
use matrix matching to select best N sets from the stored Selected CalSets;
use matrix matching to select best K samples from the N Selected CalSets;
form a calibration model with the K samples; and
apply the LAFR process to a new target sample to predict the analysis thereof.

8. Methodology according to claim 7, wherein the process parameters includes number of principal components (PCs), and Partial Least Squares (PLS) model selection.

9. Methodology according to claim 8, wherein the process parameters further include Spectral similarity measures to use for reducing the Library, and the number of samples to remain after reducing the Library.

10. Methodology according to claim 9, wherein the process parameters further include defining outlier measures to use for detecting outliers, the number of outliers to remove each iteration, and self-prediction thresholds.

11. Methodology according to claim 10, wherein the process parameters further include identifying the number of calibration sets to form, and identifying similarity measures for matrix matching.

12. Methodology according to claim 7, wherein the matrix matching comprises using the ISUₓ and ISU_{y} sample-wise similarities to mine the library of field sample spectra with reference amounts for a local training set explicitly matrix-matched to the target prediction sample, where ISUₓ and ISU_{y} sample.

13. Methodology according to claim 12, wherein ISUₓ comprises matrix-match spectra, and ISU_{y} comprises matrix-match actual and predicted analyte reference amounts which allows matching an unknown target sample analyte amount to the known analyte reference samples.

14. Methodology according to claim 13, wherein ISUₓ and ISU_{y} each comprise an indicator of system uniqueness (ISU) that is a hybrid fusion algorithm based on a plurality of similarity measures using a cross-modeling procedure, with X and Y matches, and with each sample prediction amount receiving a membership value relative to the calibration set.

15. Methodology for predicting the quantitative analysis of a target sample, comprising:
searching through a library of spectral samples with corresponding analyte values (Library X, y) using a generalized local adaptive fusion regression (LAFR) process for identifying subsets of samples with similar matrix effects;
forming linear training sets defined by the LAFR process identified subsets of samples;
forming a final local training set from the linear training sets;
forming a prediction model with the final local training set; and
using the prediction model to predict the quantitative analysis of a target sample, where the final local training set is composed of samples with the analyte amount highly similar to the unknown analyte amount in the target sample to be predicted.

16. Methodology according to claim 15, where the LAFR process further includes:
applying a decimation step to the Library to reduce the library to most N spectrally similar to target sample, and to perform an outlier check to remove reduced library components for which the target sample is an outlier;
forming linear calibration sets defined by the LAFR process parameters,
performing an outlier check to remove linear training sets for which the target sample is an outlier; and
using ISUₓ and ISU_{y} sample-wise similarities to mine the remaining linear training sets for the final local training set explicitly matrix-matched to the target prediction sample, wherein the ISUₓ and ISU_{y} sample-wise similarities comprise indicators of system uniqueness (ISU) to assess the degree of matrix matching between reference samples and the target sample.

17. Methodology according to claim 16, wherein forming linear training sets includes forming over one hundred linear local models from a library with each model representing a distinct combination of hidden matrix effects.

18. Methodology according to claim 17, wherein the ISU makes use of hybrid fusion to characterize X and Y similarities including sample-wise differences, with each sample prediction amount receiving a membership value relative to the final local training set.

19. Methodology according to claim 15, wherein each linear training set is matched to each target sample by both spectra and analyte amount.

20. Methodology according to claim 19, wherein matching parameters for spectroscopic samples include at least one of:
analyte values comprising the concentration of constituent of interest,
spectra comprising response fingerprints when externally stimulated, and
matrix effects comprising relationships between spectrum and analyte.

21. Methodology according to claim 20, wherein ISUₓ comprises a holistic characterization of implicit X sample-wise differences between target and calibration sample matrix effects, where ISUₓ values depend on sample y values.

22. Methodology according to claim 20, wherein ISU_{y} comprises a holistic characterization of implicit y sample-wise differences between target and calibration sample matrix effects, where ISU_{y} values depend on sample X values.

23. A handheld spectral device operating according to the methodology of claim 1, for making spectral measurements of a target sample in the field and predicting the quantitative analysis thereof.

24. A handheld device according to claim 23, wherein the handheld device is a smartphone remotely accessing an app for operating according to the methodology of claim 1.

25. A handheld spectral device operating according to the methodology of claim 7, for making spectral measurements of a target sample in the field and predicting the quantitative analysis thereof.

26. A handheld spectral device operating according to the methodology of claim 15, for making spectral measurements of a target sample in the field and predicting the quantitative analysis thereof.
